(19) United States
(12) Patent Application Publication
(10) Pub. No.: US 2025/0163393 A1
(43) Pub. Date: May 22, 2025

(54) METHODS AND COMPOSITIONS FOR ADVANCED BREEDING THROUGH TARGETED CHROMOSOME ENGINEERING

(71) Applicant: PIONEER HI-BRED INTERNATIONAL, INC., JOHNSTON, IA (US)

(72) Inventors: JEFFRY DAVID SANDER, ANKENY, IA (US); SERGEI SVITASHEV, JOHNSTON, IA (US); JOSHUA K. YOUNG, JOHNSTON, IA (US)

(73) Assignee: PIONEER HI-BRED INTERNATIONAL, INC., JOHNSTON, IA (US)

(21) Appl. No.: 18/840,944

(22) PCT Filed: Feb. 23, 2023
(86) PCT No.: PCT/US2023/063129

§ 371 Date: Aug. 23, 2024

Related U.S. Application Data
(60) Provisional application No. 63/314,402, filed on Feb. 26, 2022.

(51) Int. Cl. C12N 9/22; A01H 1/02; A01H 6/46; C12N 5/04

(52) U.S. Cl. CPC C12N 9/22; A01H 1/021; A01H 6/4684; C12N 5/04; C12N 2310/20; C12N 2510/00

(57) ABSTRACT

Compositions and methods are provided for large scale manipulation of genomic regions and chromosomal engineering of plant genomes that include paracentric inversions, rearrangement of pericentromeric chromosomal segments, chromosomal translocations and inversions thereof. These advanced breeding techniques provide enhanced genetic diversity within existing breeding population and increase genetic gain. Wild or outcrosses between two different crop species that can be crossed are also within scope of the disclosure. Site-specific genome manipulation tools such as CRISPR-Cas systems enable targeted chromosomal engineering of crop plants, including stable, inheritable, large-scale centromeric inversions.

### FIELD

The disclosure relates to the field of plant molecular biology, in particular, to compositions and methods for altering the genome of a cell.

### BACKGROUND

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted insertions of genes of interest in a variety of organism.

Newer technologies utilizing archaeal or bacterial adaptive immunity systems have been identified, called CRISPR (Clustered Regularly Interspaced Short Palindromic Repeats), which comprise different domains of effector proteins that encompass a variety of activities (DNA recognition, binding, and optionally cleavage).

Small-scale deletions and inversions can be common outcomes of simultaneous DSBs (double-strand breaks). The further apart the DSB points are the lower probability of ‘incorrect’ repair expected. One familiar with the field would expect the frequency of inversion to be different between tens of kilobases and tens of megabases, and thus an unexpected outcome of DSB technology.

Genetic recombination is the main source of variability and is the foundation of conventional plant breeding. However, when large chromosomal rearrangements occur, they have a significant effect on ability of homologous chromosomes to pair and recombine resulting in large number of genes to be excluded from the recombination processes. Therefore, highly efficient, controlled chromosomal rearrangements can be beneficial in many ways and have a great impact on plant breeding programs.

### SUMMARY

A method for producing an increased recombinogenic parental line of a crop plant, the method includes: identifying and targeting a low-recombinogenic pericentromeric chromosomal segment in a chromosomal arm of a crop plant cell to rearrange to a higher recombinogenic state in the chromosomal arm of the plant cell by introducing one or more site-specific DNA breaks at one or more target sites in the chromosomal segment and obtaining the modified chromosomal arm comprising the rearranged chromosomal segment that is rearranged such that the rearranged chromosomal segment is capable of increased recombination compared to a control plant cell not comprising the rearranged chromosomal segment, wherein the recombinogenic parental line is stable and the rearranged chromosomal segment is heritable in one or more progeny population. In an embodiment, the chromosomal segment is inverted such that a portion of the chromosomal segment that was positioned closer to a centromeric region in an unmodified state is closer to a telomere of the chromosomal arm in the rearranged or modified state. In an embodiment, the method further includes generating the stable, modified chromosomal arms of more than one chromosome in two or more parental lines of a breeding population for a crop breeding program, such that the modified chromosomes are recombination compatible in a breeding cross. In an embodiment, the plant comprising at least one paracentric or pericentric inversion described herein is fertile, sets seeds and the chromosomal inversion is stable and heritable in one or more progeny populations.

In an embodiment, the DNA breaks are targeted double strand breaks introduced by a site-specific nuclease selected from the group consisting of CRISPR-Cas nucleases, TALENs, meganucleases and zinc finger nucleases. In an embodiment, the chromosomal rearrangement is an inversion or a translocation. In an embodiment, the chromosomal rearrangement is within the same arm of the chromosome. In an embodiment, the rearranged or translocated chromosomal segment comprises at least about 50 kb, 75 kb, 100 kb, or 500 kb of contiguous bases. In an embodiment, the rearranged or translocated chromosomal segment is larger than about 1 Mb, 5 Mb, 10 Mb, 15 Mb, 20 Mb, 25 Mb, 50 Mb or 100 Mb.

In an embodiment, the inverted, rearranged or translocated chromosomal segment comprises one or more QTLs or one or more favorable alleles associated with an agronomic trait that were previously not associated with such traits due to low observable recombination frequency. In an embodiment, the modified chromosome or sister chromatids comprising the modified chromosomes are capable of segregating during cell division.

In an embodiment, the plant cell is a somatic cell. In an embodiment, the recombination is increased during meiotic or mitotic cell division. In an embodiment, the crop plant is selected from the group consisting of corn, soybean, cotton, canola, sorghum, wheat, rice, sunflower, alfalfa, vegetable and fruit plants.

A method of increasing recombinogenic potential of a crop breeding population, the method comprising: inverting a first chromosomal segment of at least one chromosomal arm (paracentric or paracentromeric inversion) of a first breeding line such that the inverted pericentromeric chromosomal segment is closer to the telomeric side of the chromosomal arm; inverting a second chromosomal segment of at least one chromosomal arm (paracentric or paracentromeric inversion) of a second breeding line such that the inverted pericentromeric chromosomal segment is closer to the telomeric side of the chromosomal arm; obtaining a breeding pair comprising the first breeding line having the first chromosomal segment inverted and the second breeding line having the second chromosomal segment inverted; crossing the first breeding line and the second breeding line with inverted chromosomal segments; and obtaining progeny as a result of a cross between the first breeding line and the second breeding line with inverted para-centromeric chromosomal segments.

In an embodiment, the crop is maize. In an embodiment, the chromosomal segment is at least 50 kb. In an embodiment, the chromosomal segment is larger than 1 Mb. In an embodiment, the breeding population comprises about 10% to 100% of the chromosomes comprise at least one modified chromosomal arm that includes the inverted para-centromeric chromosomal segment. In an embodiment, the inverted chromosomal segments are accomplished through targeted site-specific double strand breaks with a site-specific nuclease. In an embodiment, the inverted chromosomal segments are present in homologous chromosomes. In an embodiment, the rearrangement or translocation results in unequal inversion of the chromosomal segment.

A method for relocating a pericentromeric segment of a chromosome in a crop plant, the method comprising: introducing a first targeted site-specific break at a chromosomal arm of the chromosome, wherein the first break is present at a pericentromeric segment of the chromosomal arm and introducing a second targeted site-specific break at a chromosomal arm of a different chromosome, wherein the second break is closer to a telomeric region of the chromosomal arm; obtaining a plant with a desired translocation, wherein the pericentromeric segment is translocated towards the telomeric region of the chromosomal arm; generating progeny plants with the desired translocation; and utilizing the progeny plants in a crop breeding program for increasing genetic gain.

A method for trait introgression from a wild race to a cultivated genotype or from a genotype with the trait of interest to an elite genotype not carrying the trait, through a para-centromeric chromosomal engineering breeding approach, the method comprising: generating a recipient plant comprising a modified genotype, wherein the recipient plant comprises at least one chromosome wherein a para-centric inversion of one of the chromosomal arms is introduced by a human induced site-specific nuclease activity; obtaining a wild relative donor plant, wherein the wild relative donor plant comprises a trait of interest and is capable of being crossed with the recipient plant; performing a cross between the modified recipient plant and the donor plant to produce one or more progeny plants; and allowing chromosomal DNA transfer to occur between the donor and the recipient plant species, thereby engineering inter-chromosomal translocation and/or QTL containing DNA transfer between the donor and the recipient plants; and selecting the progeny plant comprising the trait of interest obtained from the donor plant. In an embodiment, the crop plant that comprises the para-centric or para-centromeric inversion or translocation is fertile and is capable of setting seeds.

A method for paracentric inversion of a chromosomal segment of more than one chromosome in a crop plant cell to enhance genetic recombination between homologous chromosomes during meiosis and increase genetic diversity wherein the chromosome comprises a first and second target site in the same chromosomal arm, the method comprising: introducing to the chromosome a Cas endonuclease and a first and second guide RNA, wherein the Cas endonuclease and the first and second guide RNA form a first and second complex, respectively and providing one or more oligonucleotides with substantial sequence similarity to one or more chromosomal ends at two different or distinct double strand break regions; wherein each of the first and second complexes recognizes, binds to, and cleaves the first and second target sites, respectively; incubating the cell under conditions that allow for repair of the two cleavages at the two target sites, wherein the repair results in the inversion of the segment; and validating the inversion by genotype or phenotype of the cell, or an organism that comprises the cell; wherein the segment comprises at least one million contiguous bases, wherein the first and second target sites flank the segment.

A method of increasing the efficiency of interspecific and/or intergeneric chromosomal DNA transfer, the method comprising (i) providing a donor plant species and a recipient plant species, wherein at least the recipient or the donor plant species comprise a paracentric or a pericentric inversion of at least one of the chromosomes; (ii) optionally introducing targeted double strand or single strand DNA breaks in the chromosomal regions of the donor and/or the recipient plant species such that large chromosomal fragments are targeted and providing one or more oligonucleotides with substantial sequence similarity to one or more chromosomal ends at two different or distinct double strand break regions; and (iii) allowing chromosomal DNA transfer to occur between the donor and the recipient plant species, thereby engineering interspecific and/or intergeneric chromosomal DNA transfer between the donor and the recipient plant species.

A method of reducing recombination frequency within a chromosomal segment to preserve linkage disequilibrium of one or more favorable alleles or SNPs or traits of interest in a chromosome of a crop plant, the method comprising introducing site-specific double strand breaks in at least two distant target sites of a chromosomal region, wherein the chromosomal region comprises a human introduced paracentric inversion and providing one or more oligonucleotides with substantial sequence similarity to one or more chromosomal ends at two different or distinct double strand break regions, where in the targeted double strand breaks result in an reinversion or a rearrangement of the previously translocated chromosomal segment to its original orientation such that the reinverted or rearranged chromosomal segment does not recombine or recombines at a lower frequency compared to a control plant during meiosis, wherein the reinverted or rearranged chromosomal segment comprises newly acquired or created alleles or desirable haplotypes due to increased recombination from the previous generation. In an embodiment, the inverted or rearranged chromosomal segment is in the same chromosome as the original pre-inversion/rearrangement chromosome. In an embodiment, the inverted or rearranged chromosome is in a heterologous or non-homologous chromosome. In an embodiment, the methods described herein further comprise providing to the crop plant cell at least one morphogenic factor. In an embodiment, the morphogenic factor is BBM or WUS. In an embodiment, the DNA break inducing agent is CRISPR-Cas and is provided directly to the cell as a protein.

In an embodiment, the DNA break inducing agent is CRISPR-Cas and is provided directly to the cell as an RNA molecule. In an embodiment, the DNA break inducing agent is CRISPR-Cas and is transfected/electroporated to plant protoplasts or delivered to intact plant cells with cell walls using particle bombardment. In an embodiment, the DNA break inducing agent is CRISPR-Cas and is provided directly to the crop plant cell by Agrobacterium-mediated delivery.

An engineered crop plant cell comprising a modified chromosomal arm of a crop plant chromosome, wherein a low recombinogenic para-centromeric chromosomal segment is present in a heterologous location of a higher recombinogenic state in the chromosomal arm of the plant cell and exhibits increased recombination compared to a control plant cell not comprising the rearranged chromosomal segment, during meiosis or mitosis. In an embodiment, the crop plant cell is maize. In an embodiment, the plant cell is selected from the group consisting of wheat, rice, canola, sunflower, cotton and sorghum. In an embodiment, the crop plant seed comprising the cell described herein. In an embodiment, the crop plant further includes a reinverted paracentric chromosomal segment. In an embodiment, the paracentric chromosomal segment is inverted, thereby translocating to the heterologous location. In an embodiment, the crop plant includes the cell described herein. In an embodiment, the seed is maize plant seed comprising the cell described herein. In an embodiment, the maize plant is an inbred. In an embodiment, the maize plant seed is an F1 progeny.

In an embodiment, the one or more oligonucleotides are double stranded. In an embodiment, the one or more oligonucleotides are about 50 bp to about 10,000 bp in length. Suitable lengths include for example, 100, 500, 1000, 2000, 3000, 4000, 5000, 6000, 7000, 8000, 9000, 10,000 and other longer fragments as applicable. In certain embodiments, the oligonucleotides are single stranded. In certain embodiments, the oligonucleotides contain an intervening spacer or other linker sequences to enhance chromosomal end-joining.

In an embodiment, the one or more oligonucleotides are transfected/electroporated to plant protoplasts or delivered to intact plant cells with cell walls using particle bombardment. In an embodiment, the one or more oligonucleotides are co-delivered with DSB reagents in the form of ribonucleoprotein (RNP) complexes or DNA plasmid(s). In an embodiment, the one or more oligonucleotides are delivered by a plasmid vector and flanked with the target sequences that contain a PAM. In an embodiment, the one or more oligonucleotides are released from the plasmid DNA upon the target sites cleavage by a site-directed nuclease. In an embodiment, the one or more oligonucleotides are introduced into a plant cell by Agrobacterium-mediated delivery. In an embodiment, the one or more oligonucleotides are part of a T-DNA vector and flanked with a site-directed nuclease target sites with PAM to release it from the T-DNA upon cleavage.

In an embodiment, the chromosomal rearrangement is an inversion or a reinversion (e.g., of previous chromosomal inversion event). In an embodiment, the chromosomal translocation is between two heterologous chromosomes. In an embodiment, the chromosomal translocation is between two wide-cross or interspecies chromosomes. In an embodiment, the chromosomal translocation results in duplication. In an embodiment, the chromosomal wherein the translocated segment comprises at least 50 kb of contiguous bases. In an embodiment, the translocated chromosomal segment is larger than 1 Mb. In an embodiment, the translocated chromosomal segment comprises one or more QTLs. In an embodiment, the translocated chromosomal segment comprises one or more favorable alleles associated with an agronomic trait. In an embodiment, the chromosomal translocation is present in a hybrid crop. In an embodiment, the crop plant is selected from the group consisting of corn, soybean, cotton, canola, sorghum, wheat, rice, sunflower, and alfalfa.

Provided are methods and compositions for inverting large segments of a chromosome, deleting segments of chromosomes, and relocating segments or genes or QTLs or SNPs or haplotypes using CRISPR-Cas technology (or any other DSB technology) in crop plants along with one or more oligonucleotides with substantial sequence similarity to one or more chromosomal ends at two different or distinct double strand break regions.

A method of engineering an inversion of a chromosomal segment of a chromosome in a crop plant cell, the method includes introducing to a plurality of crop plant cells, a double stand break inducing agent capable of site-specifically cleaving first and second target sites that flank the chromosomal segment of the crop plant cell, one or more oligonucleotides with substantial sequence similarity to one or more chromosomal ends at two different or distinct double strand break regions, wherein the chromosomal segment comprises at least 50 kb; incubating the cell under conditions that allow for double strand breakage and repair of the two cleavages at the two target sites, wherein inversion of the chromosomal segment occurs such that the chromosomal segment inversion is heterologous to its chromosome; regenerating a crop plant wherein the crop plant comprises the inverted chromosomal segment compared to a control crop plant; and validating the inversion by genotyping or phenotyping of the crop plant cell, or the crop plant that comprises the inverted chromosomal segment. In an embodiment, the crop plant is maize. In an embodiment, the inversion is pericentric. In an embodiment, the chromosomal segment is larger than 1 Mb. In an embodiment, the inversion is performed in a somatic cell.

A method of engineering deletion of a large chromosomal segment in a genome of a crop plant cell, wherein the chromosomal segment is characterized by at least a first and a second target site, the method includes introducing to the crop plant cell a Cas endonuclease and a first and second guide RNA and one or more oligonucleotides with substantial sequence similarity to one or more chromosomal ends at two different or distinct double strand break regions, wherein the Cas endonuclease and the first and second guide RNA form a first and second complex, respectively; wherein each of the first and second complexes recognizes, binds to, and cleaves the first and second target sites, respectively, wherein the first and second target sites flank the segment; incubating the crop plant cell under conditions that result in the removal of the chromosomal segment, wherein the segment comprises at least 100 kb; validating the deletion by genotype or phenotype of the cell, or an organism that comprises the cell; and regenerating a crop plant that does not comprise the deleted chromosomal segment.

A method for relocating a QTL on a chromosome via somatic recombination between a first and second cell, the method includes crossing the first and second cell to produce a hybrid cell, wherein the hybrid cell comprises one set of chromosomes from each of the first and second cell, wherein at least one chromosome in the hybrid cell comprises the QTL; cleaving the chromosome comprising the QTL at a target site between the QTL and the centromere of the chromosome; cleaving the chromosome homologous to the chromosome comprising the QTL at the corresponding target site; providing one or more oligonucleotides with substantial sequence similarity to one or more chromosomal ends at two different or distinct double strand break regions; reproducing the cell to obtain progeny cells, and selecting at least one progeny cell that comprises the translocation; cleaving the chromosome comprising the QTL at a target site between the QTL and the telomere; cleaving the chromosome homologous to the chromosome comprising the QTL at the corresponding target site; and validating the recombination by the genotype or phenotype of an cell or cell obtained or derived from the cell.

A method for relocating a segment of a chromosome, the method includes cleaving the first chromosome at a site desired for relocation and second chromosome at the side of the QTL closer to centromere; selecting for plants with the desired translocation; cleaving the chromosome with translocation at the end of the QTL and the normal homologous chromosome at the intended relocation site and providing one or more oligonucleotides with substantial sequence similarity to one or more chromosomal ends at two different or distinct double strand break regions; selecting for an organism with the desired translocation; selfing an organism with the desired outcome; and selecting for the progeny with 2 homologous chromosomes with the relocated QTL.

A method for a gene/QTL of interest validation and association with a specific chromosomal region, the method includes (i) generation of a hybrid between genotypes one carrying the trait and the second one without the trait that the resulting hybrid has a pair of parental chromosomes with and without trait of interest, (ii) generation of plants with hemizygous deletion of a segment of a chromosome associated with the trait of interest, wherein the deletion is influenced by one or more oligonucleotides with substantial sequence similarity to one or more chromosomal ends at two different or distinct double strand break regions, (iii) conducting agronomic evaluation of the deletion effect in the progeny plants to validate the association of the chromosomal region with the specific chromosomal segment.

A method for a fine mapping of a gene/QTL to narrow the chromosomal fragment associated with the trait, the method includes (i) generation of plants with a series of various deletions spanning the QTL region, (ii) agronomic evaluation of the plants with deletions and (iii) identification of the smallest DNA segment associated with the trait.

A method for trait introgression from a wild race to a cultivated genotype or from a genotype with the trait of interest to an elite genotype not carrying the trait, the method includes (i) generating a hybrid plant between a donor a recipient plants/genotypes, wherein the hybrid cell comprises one set of chromosomes from each of the parental plants, wherein at least one chromosome in the hybrid cell comprises the QTL, (ii) introducing targeted double strand or single strand DNA breaks in the chromosomal regions of the donor and/or the recipient plant species between the QTL and the centromere of the chromosome, cleaving the chromosome homologous to the chromosome comprising the QTL at the corresponding target site (iii) allowing chromosomal DNA transfer to occur between the donor and the recipient plant species, thereby engineering inter-chromosomal translocation and/or QTL containing DNA transfer between the donor and the recipient plants, (iv) selecting plants with desired translocation and QTL transfer, (v) introducing targeted double strand or single strand DNA breaks in the chromosomal regions of the plant cells such that chromosomal target site on the other side of the QTL of interest is targeted, (vi) allowing chromosomal DNA transfer to occur between the donor and the recipient plant species, thereby engineering inter-chromosomal translocation restoring recipient genotype chromosome now containing a gene/QTL of interest.

A method for relocating a QTL or a group of genes/QTLs of interest from their original location in a chromosome to a desired location on a different chromosome (gene/trait stacking), the method includes (i) cleaving the chromosome comprising the gene(s)/QTL(s) at a target site between the gene(s)/QTL(s) and the centromere of the chromosome, (ii) cleaving the second chromosome at the target site selected for gene(s)/QTL(s) relocation by one or more oligonucleotides with substantial sequence similarity to one or more chromosomal ends at two different or distinct double strand break regions; (iii) creating conditions allowing for inter-chromosomal translocation to occur moving gene(s)/QTL(s) into a new chromosome; (iv) selecting at least one progeny plant that comprises the translocation; (v) cleaving the chromosome comprising the gene(s)/QTL(s) at a target site between the gene(s)/QTL(s) and the telomere; (vi) cleaving the chromosome homologous to the chromosome comprising the QTL at the corresponding target site; and (vii) validating the translocation by the genotype or phenotype of a cell or cell obtained or derived from the cell; (viii) selfing (self-crossing) a plant with the desired outcome; and selecting for the progeny with 2 homologous chromosomes with the relocated gene(s)/QTL(s).

A method for inversion a segment of a chromosome in a plant cell to open genetic recombination between homologous chromosomes and increase genetic diversity wherein the chromosome comprises a first and second target site, the method includes introducing to the chromosome a Cas endonuclease and a first and second guide RNA, wherein the Cas endonuclease and the first and second guide RNA form a first and second complex, respectively; wherein each of the first and second complexes recognizes, binds to, and cleaves the first and second target sites, respectively; incubating the cell under conditions that allow for repair of the two cleavages at the two target sites and providing one or more oligonucleotides with substantial sequence similarity to one or more chromosomal ends at two different or distinct double strand break regions, wherein the repair results in the inversion of the segment; and validating the inversion by genotype or phenotype of the cell, or an organism that comprises the cell; wherein the segment comprises at least one million contiguous bases, wherein the first and second target sites flank the segment.

A method of increasing the efficiency of interspecific and/or intergeneric chromosomal DNA transfer, the method comprising (i) providing a donor plant species and a recipient plant species; (ii) introducing targeted double strand or single strand DNA breaks in the chromosomal regions of the donor and/or the recipient plant species such that large chromosomal fragments are targeted; and (iii) allowing chromosomal DNA transfer to occur between the donor and the recipient plant species by providing one or more oligonucleotides with substantial sequence similarity to one or more chromosomal ends at two different or distinct double strand break regions, thereby engineering interspecific and/or intergeneric chromosomal DNA transfer between the donor and the recipient plant species.

In an embodiment, the methods include providing to the crop plant cell at least one morphogenic factor. In an embodiment, the morphogenic factor is BBM or WUS. In an embodiment, the Cas endonuclease is provided directly to the cell as a protein. In an embodiment, the guide RNA is provided to the cell as an RNA molecule. In an embodiment, the chromosomal segment comprises at least 10 million contiguous bases.

A method of reducing recombination frequency within a chromosomal segment to preserve linkage disequilibrium of one or more favorable alleles or SNPs or traits of interest in a chromosome of a crop plant, the method comprising introducing site-specific double strand breaks in at least two distant target sites of a chromosomal region along with one or more oligonucleotides with substantial sequence similarity to one or more chromosomal ends at two different or distinct double strand break regions, where in the targeted double strand breaks result in an inversion or a rearrangement of the chromosomal segment such that the inverted or rearranged chromosomal segment does not recombine or recombines at a lower frequency compared to a control plant during meiosis. In an embodiment, the traits are transgenic traits that are present in the inverted or rearranged chromosomal segment. In an embodiment, the inverted or rearranged chromosomal segment is in the same chromosome as the original pre-inversion/rearrangement chromosome. In an embodiment, the inverted or rearranged chromosome is in a heterologous or non-homologous chromosome.

In an embodiment, the methods include a chromosomal rearrangement that is not a Robertsonian translocation. In an embodiment, the high recombinogenic state is sub-telomeric. In an embodiment, the recombinogenic potential of crop plant is further enhanced by modulating chromatic compaction of the modified parental line. In an embodiment, the chromosomal rearrangement in increased by temperature modulation.

The methods and compositions described herein are useful for modifying the genome of a cell, particularly a plant cell, and find use in a wide range of applications, for example but not limited to accelerating breeding of traits in plants.

### BRIEF DESCRIPTION OF THE DRAWINGS

The disclosure can be more fully understood from the following detailed description and the accompanying drawings, which form a part of this application.

FIG. 1 depicts a general structure of a chromosome with regions that are different in recombination ability. Pre-centromeric parts of chromosomes are known for low or no recombination, while regions closer to telomeres show high recombination activity.

FIG. 2 depict large chromosomal rearrangements leading to changes in chromosomal organization. FIG. 2A depicts large paracentric inversions in homologous chromosomes of the same species or two different species, genotypes, wild races leading to changes in chromosomal location of the corresponding regions indicated with numbers (1 and 2). As a result of paracentric inversion including the whole arm or a part of it, pre-centromeric (i.e., regions normally associated with the centromeric region) regions become sub-telomeric and vice versa. As the result, chromosomal regions previously not recombining become open for new recombination. FIG. 2B depicts large pericentric inversions in homologous chromosomes of the same species or two different species, genotypes, wild races leading to changes in chromosomal location of the corresponding regions indicated with numbers (1, 2 and 3). As a result of pericentric inversion, pre-centromeric regions become sub-telomeric and vice versa. As a result, chromosomal regions previously not recombining become open for recombination. FIG. 2C depicts chromosomal translocations in homologous chromosomes of the same species or two different species, genotypes, wild races leading to changes in chromosomal location of the corresponding regions indicated with numbers (1 and 2). As a result of the translocation including the whole arm or a part of it, pre-centromeric regions become located further away from the centromere and vice versa. Chromosomal regions that previously do not recombine or show significantly reduced recombination frequency is now available for recombination at a higher rate, thus creating more genetic diversity among existing germplasm pool.

FIG. 3 depicts crosses between same species or two different species, genotypes, or wild races carrying chromosomes with inversions (FIGS. 3A and 3B) or translocations (FIG. 3C) as depicted in FIG. 2 leading to produce or generate F1 progeny (including hybrid) plant, in which homologous chromosomes with the rearrangements described herein undergo pairing and recombination between prior pericentromeric and therefore non-recombinogenic regions indicated by the number 2 in FIG. 3A, number 3 in FIG. 3B and number 2 in FIG. 3C.

### DETAILED DESCRIPTION

In most experiments, chromosomal rearrangements (deletions, duplications, inversions, and translocations) result from a random ‘improper’ DSB repair, therefore, happen with very low frequencies. Further optimization and frequency improvement of the desired chromosomal rearrangements can be accomplished by various approaches and modifications in the experimental designs described below. Compositions and methods are described herein that enable chromosomal inversions, such as for example paracentric and pericentric. Paracentric inversions generally do not include the centromere, and often, both breakpoints occur in one arm of the chromosome. Pericentric inversions generally span the centromere, and often, there is a breakpoint in each arm of the chromosome. Illustrations for both of these chromosomal inversions are described herein.

Described here are methods for producing an increased recombinogenic parental line of a crop plant, the method includes: identifying (e.g., through bioinformatics and recombination breeding data) and selectively targeting a low-recombinogenic pericentromeric chromosomal segment in a chromosomal arm of a crop plant cell to rearrange that chromosomal segment to a higher recombinogenic state in the chromosomal arm of the plant cell by introducing one or more site-specific DNA breaks (can be double strand breaks or single strand nicks or a combination thereof) at one or more target sites in the chromosomal segment and obtaining the modified chromosomal arm comprising the rearranged chromosomal segment that is rearranged such that the rearranged chromosomal segment is capable of increased recombination within that portion compared to a control plant cell not comprising the rearranged chromosomal segment, wherein the recombinogenic parental line is stable and the rearranged chromosomal segment is heritable in one or more progeny population. Similarly, other parental lines with centromeric inversions are generated that provide a significant increase in germplasm diversity in the progeny plants where the modified parental lines are derived from an existing breeding population.

In certain embodiment, a transgenic loci with one or more transgenic traits e.g., herbicide tolerance trait and insect resistance traits in the sub-telomeric or telomeric segment is rearranged such that the transgenic loci is less recombinogenic and therefore is easier to breed where the transgenic traits within the transgenic loci segregate and the linkage is not readily broken since the rearrangement segment is in the pericentromeric location.

In certain embodiments, the centromeric chromosomal rearrangements including inversions and translocations described herein are enhanced by using one or more factors involved in recombination. Examples include Spol1 polynucleotide or polypeptide (exogenous or endogenous), a Rec8 (aka Afd1 in maize) (exogenous or endogenous) polynucleotide or polypeptide, an OSD1-1A (exogenous or endogenous) polynucleotide or polypeptide, and an OSD1-3A (exogenous or endogenous) polynucleotide or polypeptide, and RecQ helicases.

In certain embodiments, genomic architecture is modulated such that effective chromosome recognition, pairing, and recombination are facilitated. Chromosomal structures including telomeres and subtelomeres during meiosis at chromosome recognition and pairing are modulated to permit chromosome recombination between homologs (equivalent chromosomes in the same genome) during centromeric inversions and rearrangements disclosed herein.

In certain embodiments, the plant species are plant polyploids and terminal chromosome regions are manipulated to increase inversion frequency or percentage between homologous chromosomes and recognition of homoeologs (equivalent chromosomes from related genomes). Generally, the DNA sequence of telomeres present in the chromosomes is not chromosome-specific and therefore such telomeres can be manipulated across multiple chromosomes at the same time for chromosomal segment rearrangement.

In certain embodiment, for plant breeding purposes, chromosome manipulation in hybrids or interspecific genetic crosses are provided. For example, recombination in interspecific chromosome associations is increased to enable transfer of desirable agronomic traits from related genetic donor species (e.g., wild-relative) into cultivated crop species.

In an embodiment, the chromosomal segment is inverted such that a portion of the chromosomal segment that was positioned closer to a centromeric region in an unmodified state is closer to a telomere of the chromosomal arm in the rearranged or modified state. In an embodiment, the method further includes generating the stable, modified chromosomal arms of more than one chromosome in two or more parental lines of a breeding population for a crop breeding program, such that the modified chromosomes are recombination compatible in a breeding cross. In an embodiment, the plant comprising at least one paracentric or pericentric inversion described herein is fertile, sets seeds and the chromosomal inversion is stable and heritable in one or more progeny populations.

Methods for translocating a large chromosomal segment in a crop plant cell are described herein. These methods include for example, where the chromosomal segment is flanked by at least a first and a second genomic target site and include introducing to a plurality of crop plant cells, a Cas endonuclease and a first and second guide RNA, wherein the Cas endonuclease and the first and second guide RNA form a first and second complex, respectively; wherein each of the first and second complexes recognize, bind to, and cleave the first and second target sites, respectively; incubating the crop plant cells under conditions that result in translocation of the chromosomal segment; regenerating a crop plant wherein the crop plant comprises the translocated chromosomal segment compared to a control crop plant; and validating the chromosomal translocation by genotype or phenotype of the crop plant cell or the crop plant. In an embodiment, the Cas endonuclease is Cas9, Cas12a, Cas12f or a combination thereof. Suitable conditions to induce large chromosomal translocations include selecting appropriate target sites, effective amount of the Cas endonuclease (e.g., expression levels if expressed from a transcript or protein concentration is provided as ribonucleoprotein (RNP) complexes), type, amount and nature of the guide RNAs, efficiency of the Cas endonuclease and the type of tissue used for transformation or introducing the DSB components (e.g., embryo tissue, vegetative tissue such leaf cells).

Methods of engineering an inversion of a chromosomal segment of a chromosome in a crop plant cell are described herein. These methods include for example, introducing to a plurality of crop plant cells, a double stand break inducing agent (e.g., Cas endonucleases) capable of site-specifically cleaving first and second target sites that flank the chromosomal segment of the crop plant cell. Large chromosomal segments comprise at least 50 kb, but can vary in length depending on the genomic context and genomic chromosomal architecture. For example, if multiple haplotypes reside in a genomic window of 1 Mb to about 100 Mb, that genomic regions are targets for chromosomal engineering, which includes inversion, translocation, or deletion if such haplotypes are deleterious and their removal improves genetic gain. Various regions of the chromosomes are suitable for chromosomal engineering. Centromeric, peri-centric or peri-centromeric, or telomeric or sub-telomeric regions are suitable for engineering inversions, translocations, controlled crossovers, controlled recombination, or other large-scale genome manipulation contemplated herein for crop plant. In an embodiment, the inversion is pericentric. In an embodiment, the chromosomal segment is larger than 1 Mb. In an embodiment, the inversion is performed in a somatic cell

Terms used in the claims and specification are defined as set forth below unless otherwise specified. It must be noted that, as used in the specification and the appended claims, the singular forms “a,” “an” and “the” include plural referents unless the context clearly dictates otherwise.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell. “Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY-5, WINDOW=4 and DIAGONALS SAVED-4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY-0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, CA) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. An RNA transcript is referred to as the mature RNA or mRNA when it is an RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In certain preferred embodiments, host cell is a plant cell including crop plant cell.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre- or propeptides present in the primary translation product have been removed).

“Precursor” protein refers to the primary product of translation of mRNA (i.e., with pre- and propeptides still present). Pre- and propeptides may be but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes but is not limited to: a Cas9 protein, a Cpf1 (Cas12a) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. A Cas endonuclease described herein comprises one or more nuclease domains. The endonucleases of the disclosure may include those having one or more RuvC nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity.

A “Cas endonuclease” may comprise domains that enable it to function as a double-strand-break-inducing agent. A “Cas endonuclease” may also comprise one or more modifications or mutations that abolish or reduce its ability to cleave a double-strand polynucleotide (dCas). In some aspects, the Cas endonuclease molecule may retain the ability to nick a single-strand polynucleotide (for example, a D10A mutation in a Cas9 endonuclease molecule) (nCas9).

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double-strand break in) the target site is retained. The portion or subsequence of the Cas endonuclease can comprise a complete or partial (functional) peptide of any one of its domains such as for example, but not limiting to a complete of functional part of a Cas3 HD domain, a complete of functional part of a Cas3 Helicase domain, complete of functional part of a Cascade protein (such as but not limiting to a Cas5, Cas5d, Cas7 and Cas8b1).

The terms “functional variant”, “variant that is functionally equivalent” and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a cascade (comprises at least a second protein domain that can form a cascade with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “cascade” and “cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be an RNA sequence, a DNA sequence, or a combination thereof (an RNA-DNA combination sequence).

The terms “functional fragment”, “fragment that is functionally equivalent” and “functionally equivalent fragment” of a guide RNA, crRNA or tracrRNA are used interchangeably herein, and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, an RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, an RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence, “target DNA”, “target locus”, “genomic target site”, “genomic target sequence”, “genomic target locus”, “target polynucleotide”, and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The term “engineering” used in the context of chromosomal engineering generally refers to induced or introduced chromosomal changes that are not spontaneous or naturally occurring changes to a chromosome or a chromosomal segment. For example, introducing DNA breaks in a region of a chromosome through an introduced nuclease that is site-specific or otherwise targets the chromosome in a controlled or defined manner as opposed to non-specific natural variations introduced to the genome during, for example, a breeding process.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The term “plant-optimized Cas endonuclease” herein refers to a Cas protein, including a multifunctional Cas protein, encoded by a nucleotide sequence that has been optimized for expression in a plant cell or plant.

A “plant-optimized nucleotide sequence encoding a Cas endonuclease”, “plant-optimized construct encoding a Cas endonuclease” and a “plant-optimized polynucleotide encoding a Cas endonuclease” are used interchangeably herein and refer to a nucleotide sequence encoding a Cas protein, or a variant or functional fragment thereof, that has been optimized for expression in a plant cell or plant. A plant comprising a plant-optimized Cas endonuclease includes a plant comprising the nucleotide sequence encoding for the Cas sequence and/or a plant comprising the Cas endonuclease protein. In one aspect, the plant-optimized Cas endonuclease nucleotide sequence is a maize-optimized, rice-optimized, wheat-optimized, soybean-optimized, cotton-optimized, or canola-optimized Cas endonuclease.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of a plant.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

The term “non-conventional yeast” herein refers to any yeast that is not a Saccharomyces (e.g., S. cerevisiae) or Schizosaccharomyces yeast species. (see “Non-Conventional Yeasts in Genetics, Biochemistry and Biotechnology: Practical Protocols”, K. Wolf, K. D. Breunig, G. Barth, Eds., Springer-Verlag, Berlin, Germany, 2003).

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos may be separated into two different groups, one receiving a treatment (such as the introduction of a CRISPR-Cas effector endonuclease) and one control that does not receive such treatment. Any phenotypic differences between the two groups may thus be attributed solely to the treatment and not to any inherency of the plant's endogenous genetic makeup.

“Introducing” is intended to mean presenting to a target, such as a cell or organism, a polynucleotide or polypeptide or polynucleotide-protein complex, in such a manner that the component(s) gains access to the interior of a cell of the organism or to the cell itself.

A “polynucleotide of interest” includes any nucleotide sequence encoding a protein or polypeptide that improves desirability of crops, i.e. a trait of agronomic interest. Polynucleotides of interest: include, but are not limited to, polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

A “complex trait locus” includes a genomic locus that has multiple transgenes genetically linked to each other.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

The meaning of abbreviations is as follows: “sec” means second(s), “min” means minute(s), “h” means hour(s), “d” means day(s), “uL” means microliter(s), “mL” means milliliter(s), “L” means liter(s), “uM” means micromolar, “mM” means millimolar, “M” means molar, “mmol” means millimole(s), “umole” or “umole” mean micromole(s), “g” means gram(s), “ug” or “ug” means microgram(s), “ng” means nanogram(s), “U” means unit(s), “bp” means base pair(s) and “kb” means kilobase(s).

### Double-Strand-Break (DSB) Inducing Agents (DSB Agents)

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, DC)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186 (2): 757-61 and Boch et al., (2009), Science 326 (5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. WO2007/025097 application published Mar. 1, 2007).

In addition to the double-strand break inducing agents, site-specific base conversions can also be achieved to engineer one or more nucleotide changes to create one or more EMEs described herein into the genome. These include for example, a site-specific base edit mediated by an C·G to T·A or an A·T to G·C base editing deaminase enzymes (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016): 420-4.

Any double-strand-break or -nick or -modification inducing agent may be used for the methods described herein, including for example but not limited to: Cas endonucleases, recombinases, TALENs, zinc finger nucleases, restriction endonucleases, meganucleases, and deaminases.

### CRISPR Systems and Cas Endonucleases

Methods and compositions are provided for polynucleotide modification with a CRISPR Associated (Cas) endonuclease. Class I Cas endonucleases comprise multisubunit effector complexes (Types I, III, and IV), while Class 2 systems comprise single protein effectors (Types II, V, and VI) (Makarova et al. 2015, Nature Reviews Microbiology Vol. 13:1-15; Zetsche et al., 2015, Cell 163, 1-13; Shmakov et al., 2015, Molecular Cell 60, 1-13; Haft et al., 2005, Computational Biology, PLOS Comput Biol 1 (6): e60; and Koonin et al. 2017, Curr Opinion Microbiology 37:67-78). In Class 2 Type II systems, the Cas endonuclease acts in complex with a guide RNA (gRNA) that directs the Cas endonuclease to cleave the DNA target to enable target recognition, binding, and cleavage by the Cas endonuclease. The gRNA comprises a Cas endonuclease recognition (CER) domain that interacts with the Cas endonuclease, and a Variable Targeting (VT) domain that hybridizes to a nucleotide sequence in a target DNA. In some aspects, the gRNA comprises a CRISPR RNA (crRNA) and a trans-activating CRISPR RNA (tracrRNA) to guide the Cas endonuclease to its DNA target. The crRNA comprises a spacer region complementary to one strand of the double strand DNA target and a region that base pairs with the tracrRNA, forming an RNA duplex. In some aspects, the gRNA is a “single guide RNA” (sgRNA) that comprises a synthetic fusion of crRNA and tracrRNA. In many systems, the Cas endonuclease-guide polynucleotide complex recognizes a short nucleotide sequence adjacent to the target sequence (protospacer), called a “protospacer adjacent motif” (PAM).

Examples of a Cas endonuclease include but are not limited to Cas9 and Cpf1. Cas9 (formerly referred to as Cas5, Csn1, or Csx12) is a Class 2 Type II Cas endonuclease (Makarova et al. 2015, Nature Reviews Microbiology Vol. 13:1-15). A Cas9-gRNA complex recognizes a 3′ PAM sequence (NGG for the S. pyogenes Cas9) at the target site, permitting the spacer of the guide RNA to invade the double-stranded DNA target, and, if sufficient homology between the spacer and protospacer exists, generate a double-strand break cleavage. Cas9 endonucleases comprise RuvC and HNH domains that together produce double strand breaks, and separately can produce single strand breaks. For the S. pyogenes Cas9 endonuclease, the double-strand break leaves a blunt end. Cpf1 is a Class 2 Type V Cas endonuclease, and comprises nuclease RuvC domain but lacks an HNH domain (Yamane et al., 2016, Cell 165:949-962). Cpf1 endonucleases create “sticky” overhang ends.

Some uses for Cas9-gRNA systems at a genomic target site include but are not limited to insertions, deletions, substitutions, or modifications of one or more nucleotides at the target site; modifying or replacing nucleotide sequences of interest (such as a regulatory elements); insertion of polynucleotides of interest; gene knock-out; gene-knock in; modification of splicing sites and/or introducing alternate splicing sites; modifications of nucleotide sequences encoding a protein of interest; amino acid and/or protein fusions; and gene silencing by expressing an inverted repeat into a gene of interest.

In some aspects, a “polynucleotide modification template” is provided that comprises at least one nucleotide modification when compared to the nucleotide sequence to be edited. A nucleotide modification can be at least one nucleotide substitution, addition, deletion, or chemical alteration. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In some aspects, a polynucleotide of interest is inserted at a target site and provided as part of a “donor DNA” molecule. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome. The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963). The amount of homology or sequence identity shared by a target and a donor polynucleotide can vary and includes total lengths and/or regions.

The process for editing a genomic sequence at a Cas9-gRNA double-strand-break site with a modification template generally comprises: providing a host cell with a Cas9-gRNA complex that recognizes a target sequence in the genome of the host cell and is able to induce a single- or double-strand-break in the genomic sequence, and optionally at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the double-strand break. Genome editing using double-strand-break-inducing agents, such as Cas9-gRNA complexes, has been described, for example in US20150082478 published on 19 Mar. 2015, WO2015026886 published on 26 Feb. 2015, WO2016007347 published 14 Jan. 2016, and WO2016025131 published on 18 Feb. 2016.

To facilitate optimal expression and nuclear localization for eukaryotic cells, the gene comprising the Cas endonuclease may be optimized as described in WO2016186953 published 24 Nov. 2016, and then delivered into cells as DNA expression cassettes by methods known in the art. In some aspects, the Cas endonuclease is provided as a polypeptide. In some aspects, the Cas endonuclease is provided as a polynucleotide encoding a polypeptide. In some aspects, the guide RNA is provided as a DNA molecule encoding one or more RNA molecules. In some aspects, the guide RNA is provide as RNA or chemically-modified RNA. In some aspects, the Cas endonuclease protein and guide RNA are provided as a ribonucleoprotein complex (RNP).

Once a double-strand break is induced in the genome, cellular DNA repair mechanisms are activated to repair the break.

### Double-Strand-Break Repair and Polynucleotide Modification

A double-strand-break-inducing agent, such a guided Cas endonuclease can recognize, bind to a DNA target sequence and introduce a single strand (nick) or double-strand break. Once a single or double-strand break is induced in the DNA, the cell's DNA repair mechanism is activated to repair the break, for example via nonhomologous end-joining (NHEJ) or Homology-Directed Repair (HDR) processes which can lead to modifications at the target site.

SDN1 covers the application of a SDN (site-directed nuclease) without an additional donor DNA or repair template. Thus, the editing outcome depends on the DSB repair pathway of the plant genome. As the predominant DSB repair pathway is NHEJ, small insertions or deletions can occur (SDNla). In the case of tandemly arranged SDNs, larger deletions can be obtained (SDN1b). Furthermore, inversions (SDN1c) or translocations (SDN1d) can be generated by multiplexed SDN1 approaches.

SDN2 describes the use of a SDN with an additional DNA “polynucleotide modification template” to introduce small mutations in a controlled manner. Here, a template mainly homologous to the target sequence is provided to be the substrate for HR-mediated DSB repair following the induction of one or two adjacent DSBs. This approach allows the introduction of small mutations that could also occur naturally, per se.

SDN3 describes the use of a SDN with an additional “donor polynucleotide” or “donor DNA” to introduce large stretches of exogenous DNA at a pre-determined locus, adding or replacing genetic information. Mechanistically, this process relies on HR-mediated DSB repair like SDN2, and the discrimination is arbitrary as the size of the sequence inserted can vary significantly.

Both SDN2 and SDN3 are types of homology-directed repair (HDR) of a double-strand break in a polynucleotide, and involve methods of introducing a heterologous polynucleotide as either a template for repair of the double strand break (SDN2), or insertion of a new double-stranded polynucleotide at the double strand break site (SDN3). SDN2 repairs may be detected by the presence of one or a few nucleotide changes (mutations). SDN3 repairs may be detected by the presence of a novel contiguous heterologous polynucleotide.

Modification of a target polynucleotide includes any one or more of the following: insertion of at least one nucleotide, deletion of at least one nucleotide, chemical alteration of at least one nucleotide, replacement of at least one nucleotide, or mutation of at least one nucleotide. In some aspects, the DNA repair mechanism creates an imperfect repair of the double-strand break, resulting in a change of a nucleotide at the break site. In some aspects, a polynucleotide template may be provided to the break site, wherein the repair results in a template-directed repair of the break. In some aspects, a donor polynucleotide may be provided to the break site, wherein the repair results in the incorporation of the donor polynucleotide into the break site.

In some aspects, the methods and compositions described herein improve the probability of a non-NHEJ repair mechanism outcome at a DSB. In one aspect, an increase of the HDR to NHEJ repair ratio is effected.

### Homology-Directed Repair and Homologous Recombination

Homology-directed repair (HDR) is a mechanism in cells to repair double-stranded and single stranded DNA breaks. Homology-directed repair includes homologous recombination (HR) and single-strand annealing (SSA). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions.

Alteration of the genome of a prokaryotic and eukaryotic cell or organism cell, for example, through homologous recombination (HR), is a powerful tool for genetic engineering.

### Improving the Probability of HDR in DSB Repair

Several methods for encouraging the repair of a double strand break via HDR are contemplated, based on the facts that (1) Cas9 has a high affinity for, and is slow to release, its cleaved substrate (Richardson, C. et al. (2016) Nat. Biotechnol. 34:339-344); and (2) the observation by the inventors that the mutation outcomes for polynucleotide cleavage are often non-random and reproducible (unpublished). The inventors have conceived that retargeting a polynucleotide double-strand-break site, providing multiple opportunities for DSB repair, encourages the occurrence of HDR (e.g., HR) vs NHEJ. The inventors have also conceived that because recombinogenic intermediates involve 3′ overhangs, additional single strand breaks flanking the double-strand break site will produce destabilized duplexes, leading to a recombinogenic intermediate. In some cases, different endonucleases (e.g., from different source organisms or CRISPR loci, or engineered enzymes, or nickases) are used.

In some aspects, the fraction or percent of HR reads is greater than of a comparator, such as a control sample, sample with NHEJ repair, or as compared to the total mutant reads. In some aspects, the fraction or percent of HR reads is greater than of the control sample (no DSB agent). In some aspects, the fraction or percent of HR reads is greater than the fraction or percent of NHEJ reads. In some aspects, the fraction or percent of HR reads is greater than the fraction or percent of total mutant reads (NHEJ+HR).

In some aspects, the fraction of HR reads relative to a comparator is at least 2, 3, 4, 5, 6, 7, 8, 9, 10, between 10 and 15, 15, between 15 and 20, 20, between 20 and 25, 25, between 25 and 30, 30, between 30 and 40, 40, between 40 and 50, 50, between 50 and 60, 60, between 60 and 70, 70, between 70 and 80, 80, between 80 and 90, 90, between 90 and 100, 100, between 100 and 125, 125, between 125 and 150, greater than 150, or infinitely greater.

In some aspects, the percent of HR reads relative a comparator is at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 20%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% greater.

In one aspect of the method, a double-strand-break is created, repaired, and recurrently cleaved by any method or composition, for example but not limited to a Cas endonuclease and guide RNA. Briefly, a DSB inducing agent (e.g., Cas endonuclease and first guide RNA) recognize, bind to, and cleave a target polynucleotide. A first double-strand-break is created, and repaired. In some aspects, the repair results in a change of the target site polynucleotide sequence (for example, but not limited to, an insertion of a nucleotide, a deletion of a nucleotide, or a replacement of a nucleotide). In some aspects, a repair template is provided for a specific target polynucleotide repair composition outcome. In this case, the repair template is flanked with inverted target site (PAM on the inside). A second guide RNA is introduced that is complementary to the mutation that was created by the repair by the first double-strand break. In some aspects, the DSB repair composition outcome is determined by the introduction of a donor polynucleotide template or insertion, and the second guide RNA designed to be complementary to that determined target sequence outcome. In some aspects, the second guide RNA is designed to be complementary to the most commonly created repair mutation. In some aspects, the second guide RNA is designed to be complementary to a desired DNA repair outcome. In some aspects, a library of second guide RNAs is designed that are complementary to all possible mutations of the target site. The mutation(s) created by the first double-strand-break repair may be either known or predicted bioinformatically. The second guide RNA acts in concert with the Cas endonuclease (either provided de novo or the same Cas endonuclease that was present for the first DSB) to create a second double-strand-break at the same site (within the on-target recognition sequence of the Cas endonuclease/first guide RNA complex). In some aspects, instead of a second guide RNA and a Cas endonuclease creating the second DSB, another DSB inducing reagent may be introduced. The second DSB has a higher probability of repair by HDR than NHEJ, as compared to the repair of the first DSB (i.e., the probability of HDR is increased, or the frequency of HDR is increased, or the ratio of HDR to NHEJ is increased). In general, there is a subsequent cut at a previous cut site, which in some aspects can be accomplished by the introduction of another Cas endonuclease/gRNA complex. Continued cleavage in a sequential manner increases the frequency of HDR as a DSB repair mechanism.

In one aspect of the method, a double-strand-break is created, repaired, and recursively cleaved by any method or composition, for example but not limited to a Cas endonuclease and guide RNA. Briefly, a DSB inducing agent (e.g., Cas endonuclease and first guide RNA) recognize, bind to, and cleave a target polynucleotide. The first guide RNA is provided as a DNA sequence on a plasmid that further comprises a spacer sequence. In some aspects, the DNA encoding the gRNA is operably linked to a regulatory expression element. A first double-strand-break is created, and repaired. The composition of the repaired target polynucleotide is used as the basis of a mutation generated by Cas editing of the spacer on the plasmid comprising the gRNA DNA and spacer. The mutated spacer composition directs the generation of a second gRNA that is complementary to the sequence of the repaired targeted polynucleotide of the first DSB, and a second double-strand break is induced at the target site by the Cas endonuclease and the second gRNA. The cycle may then repeat, with sequence of the newly repaired second DSB then being used as a template for the composition of a third gRNA that is complementary to the sequence of the repaired second DSB polynucleotide, and so forth. In this manner a loop of DSB generation and repair occurs, with each subsequent repair after the first having a higher probability of repair via HDR than NHEJ, as compared to the mechanism of the first repair. The process may stop by any of a number of methods, including but not limited to: titrated reagent availability, mutation induced in the region of the gRNA DNA expression construct that renders the expression cassette or transcribed gRNA to be non-functional, an external factor that may optionally be inducible or repressible, or via the introduction of another molecule.

In one aspect of the method, a nick (cleavage of double-stranded DNA on only one of the two phosphate backbones) is created adjacent to a double-strand-break on a target polynucleotide. In one variation of this aspect a single nick is created. In one variation of this aspect, two nicks are created. In one variation of this aspect, two nicks are created, one each flanking the two sides of the DSB. In one embodiment, the double-strand-break is created by one Cas endonuclease, and the nick(s) is (are) created by a different molecule (e.g., a molecule derived from a different organism, or a Cas endonuclease that lacks double strand break creation functionality but possesses nickase activity (for example, nCas9)). Due to the presence of adjacent nick(s), double-strand-break repair of the DSB at the target site has a higher probability of being repaired by HDR than by NHEJ, or has a higher frequency of HDR as compared to a DSB at the same locus that does not have one or more nicks adjacent to the DSB. In some aspects, the distance between the nick and the DSB site is 10 basepairs, between 10 and 20 basepairs, 20 basepairs, between 20 and 30 basepairs, 30 basepairs, between 30 and 40 basepairs, 40 basepairs, between 40 and 50 basepairs, 50 basepairs, between 50 and 60 basepairs, 60 basepairs, between 60 and 70 basepairs, 70 basepairs, between 70 and 80 basepairs, 80 basepairs, between 80 and 90 basepairs, 90 basepairs, between 90 and 100 basepairs, 100 basepairs, between 100 and 110 basepairs, 110 basepairs, between 110 and 120 basepairs, or greater than 120 basepairs in length.

In addition to improving the probability of an HDR repair mechanism outcome, other DNA repair outcomes that are contemplated to be improved with the methods described herein include gene targeting, gene editing, gene drop-out, gene swap (deletion plus insertion), and promoter swap (deletion plus insertion).

### Gene Targeting

The compositions and methods described herein can be used for gene targeting.

In general, DNA targeting can be performed by cleaving one or both strands at a specific polynucleotide sequence in a cell with a Cas endonuclease associated with a suitable guide polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The length of the DNA sequence at the target site can vary, and includes, for example, target sites that are at least 12, 13, 14, 15, 16, 17, 18, 19, 20, 21, 22, 23, 24, 25, 26, 27, 28, 29, 30, or more than 30 nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease.

Assays to measure the single or double-strand break of a target site by an endonuclease are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

### Gene Editing

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO/2016/025131 published on 18 Feb. 2016.

Some uses for guide RNA/Cas endonuclease systems have been described (see for example: US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene drop-out, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

In one embodiment, the invention describes a method for modifying a target site in the genome of a cell, the method comprising introducing into a cell at least one Cas endonuclease and guide RNA, and identifying at least one cell that has a modification at the target site.

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Polynucleotides of interest and/or traits can be stacked together in a complex trait locus as described in WO2012129373 published 27 Sep. 2012, and in WO2013112686, published 1 Aug. 2013. The guide polynucleotide/Cas9 endonuclease system described herein provides for an efficient system to generate double-strand breaks and allows for traits to be stacked in a complex trait locus.

A guide polynucleotide/Cas system as described herein, mediating gene targeting, can be used in methods for directing heterologous gene insertion and/or for producing complex trait loci comprising multiple heterologous genes in a fashion similar as disclosed in WO2012129373 published 27 Sep. 2012, where instead of using a double-strand break inducing agent to introduce a gene of interest, a guide polynucleotide/Cas system as disclosed herein is used. By inserting independent transgenes within 0.1, 0.2, 0.3, 0.4, 0.5, 1.0, 2, or even 5 centimorgans (cM) from each other, the transgenes can be bred as a single genetic locus (see, for example, US20130263324 published 3 Oct. 2013 or WO2012129373 published 14 Mar. 2013). After selecting a plant comprising a transgene, plants comprising (at least) one transgenes can be crossed to form an F1 that comprises both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Further uses for guide RNA/Cas endonuclease systems have been described (See for example: US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Resulting characteristics from the gene editing compositions and methods described herein may be evaluated. Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for a particular trait. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

### Chromosomal Rearrangements

Breaks in chromosomes are usually repaired restoring the original order of the chromatin. As multiple breaks can occur simultaneously, in some rare cases their repair maybe incorrect resulting in various chromosomal aberrations such as deletions and inversions (if both DSBs are in the same chromosome) or in translocations involving fragments of different chromosomes. All these rearrangements, although rare, are known to occur naturally and well documented for both mammalian and plant species. Those random alterations most frequently have deleterious effects on wellbeing and lives of mammalian species. Due to the great genome plasticity and adaptation, plant species can often withstand and accumulate such rearrangements.

Genetic recombination is the main source of variability and is the foundation of conventional plant breeding. However, when large chromosomal rearrangements occur, they have a significant effect on ability of homologous chromosomes to pair and recombine resulting in large number of genes to be excluded from the recombination processes. Therefore, controlled chromosomal rearrangements, restoring the natural order of chromatin, can be beneficial in many ways and have a great impact on plant breeding programs. For example, the ability to restore chromosomes with inversions will open those regions for recombination. Alternatively, if certain regions contain important trait genes and their preservation is preferred, inversion of this region will prevent recombination and conserve the desirable genotype.

In addition, deletions of different sizes may be used to map important genes and quantitative trait loci (QTLs) while inter-chromosomal translocations can be used to move desirable traits from wild races to elite genotypes without deleterious effects associated with linkage drag. Targeted translocations may also allow moving large chromosomal fragments containing important QTLs within the genome. Below we provide detailed descriptions of these types of chromosomal rearrangements and examples of how CRISPR-Cas technology can be used to facilitate desirable outcomes opening new opportunities for plant breeding programs.

Many types of chromosomal rearrangements may be accomplished with the methods and compositions provided herein, such as, but not limited to: large deletions, large inversions, and gene relocation. Double strand break agents in association with other modifiers of recombination such as, epigenetic modifiers, provide additional tools to induce targeted, high-frequency chromosomal crossovers, translocations and inversions. These targeted recombination events increase genetic gain and provide additional diversity.

Pericentromeric or pericentric generally refers to regions of a chromosome on either side of the centromere.

In some aspects, the chromosomal segment is at least about 1 kb, between 1 kb and 10 kb, at least about 10 kb, between 10 kb and 20 kb, at least about 20 kb, between 20 kb and 30 kb, at least about 30 kb, between 30 kb and 40 kb, at least about 40 kb, between 40 kb and 50 kb, at least about 50 kb, between 50 kb and 60 kb, at least about 60 kb, between 60 kb and 70 kb, at least about 70 kb, between 70 kb and 80 kb, at least about 80 kb, between 80 kb and 90 kb, at least about 90 kb, between 90 kb and 100 kb, or greater than 100 kb. In some aspects, the segment is at least about 100 kb, between 100 kb and 150 kb, at least about 150 kb, between 150 kb and 200 kb, at least about 200 kb, between 200 kb and 250 kb, at least about 250 kb, between 250 kb and 300 kb, at least about 300 kb, between 300 kb and 350 kb, at least about 350 kb, between 350 kb and 400 kb, at least about 400 kb, between 400 kb and 450 kb, at least about 450 kb, between 450 kb and 500 kb, at least about 500 kb, between 500 kb and 550 kb, at least about 550 kb, between 550 kb and 600 kb, at least about 600 kb, between 600 kb and 650 kb, at least about 650 kb, between 650 kb and 700 kb, at least about 700 kb, between 700 kb and 750 kb, at least about 750 kb, between 750 kb and 800 kb, at least about 800 kb, between 800 kb and 850 kb, at least about 850 kb, between 850 kb and 900 kb, at least about 900 kb, between 900 kb and 950 kb, at least about 950 kb, between 950 kb and 1000 kb, at least about 1000 kb, between 1000 kb and 1050 kb, at least about 1050 kb, between 1050 kb and 1100 kb, or greater than 1100 kb. In some aspects, the segment is at least about 1 Mb, between 1 Mb and 10 Mb, at least about 10 Mb, between 10 Mb and 20 Mb, at least about 20 Mb, between 20 Mb and 30 Mb, at least about 30 Mb, between 30 Mb and 40 Mb, at least about 40 Mb, between 40 Mb and 50 Mb, at least about 50 Mb, between 50 Mb and 60 Mb, at least about 60 Mb, between 60 Mb and 70 Mb, at least about 70 Mb, between 70 Mb and 80 Mb, at least about 80 Mb, between 80 Mb and 90 Mb, at least about 90 Mb, between 90 Mb and 100 Mb, or greater than 100 Mb

### Recombinant Constructs and Transformation of Cells

The disclosed guide polynucleotides, Cas endonucleases, polynucleotide modification templates, donor DNAs, guide polynucleotide/Cas endonuclease systems disclosed herein, and any one combination thereof, optionally further comprising one or more polynucleotide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, NY (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

### Components for Expression and Utilization of CRISPR-Cas Systems in Prokaryotic and Eukaryotic Cells

The invention further provides expression constructs for expressing in a prokaryotic or eukaryotic cell/organism a guide RNA/Cas system that is capable of recognizing, binding to, and optionally nicking, unwinding, or cleaving all or part of a target sequence.

In one embodiment, the expression constructs of the disclosure comprise a promoter operably linked to a nucleotide sequence encoding a Cas gene (or plant optimized, including a Cas endonuclease gene described herein) and a promoter operably linked to a guide RNA of the present disclosure. The promoter is capable of driving expression of an operably linked nucleotide sequence in a prokaryotic or eukaryotic cell/organism.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al., Nucleic Acids Res. 41:4336-4343; Ma et al., Mol. Ther. Nucleic Acids 3: e161). This strategy has been successfully applied in cells of several different species including maize and soybean (US20150082478 published 19 Mar. 2015). Methods for expressing RNA components that do not have a 5′ cap have been described (WO2016/025131 published 18 Feb. 2016).

Various methods and compositions can be employed to obtain a cell or organism having a polynucleotide of interest inserted in a target site for a Cas endonuclease. Such methods can employ homologous recombination (HR) to provide integration of the polynucleotide of interest at the target site. In one method described herein, a polynucleotide of interest is introduced into the organism cell via a donor DNA construct.

The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity at least of about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

### Polynucleotides of Interest

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in traits of agronomic interest such as but not limited to: crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72);

Polynucleotides of interest includes genes that can be stacked or used in combination with other traits, such as but not limited to herbicide resistance or any other trait described herein. Polynucleotides of interest and/or traits can be stacked together in a complex trait locus as described in US20130263324 published 3 Oct. 2013 and in WO/2013/112686, published 1 Aug. 2013. A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, US20090133152 published 21 May 2009. The method also comprises recovering a plant from the plant cell comprising a polynucleotide of interest integrated into its genome. The plant may be sterile or fertile. It is recognized that any polynucleotide of interest can be provided, integrated into the plant genome at the target site, and expressed in a plant.

### Optimization of Sequences for Expression in Plants

These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

### Expression Elements

Any polynucleotide encoding a Cas protein, other CRISPR system component, or other polynucleotide disclosed herein may be functionally linked to a heterologous expression element, to facilitate transcription or regulation in a host cell. Such expression elements include but are not limited to: promoter, leader, intron, and terminator. Expression elements may be “minimal”-meaning a shorter sequence derived from a native source, that still functions as an expression regulator or modifier. Alternatively, an expression element may be “optimized”-meaning that its polynucleotide sequence has been altered from its native state in order to function with a more desirable characteristic in a particular host cell (for example, but not limited to, a bacterial promoter may be “maize-optimized” to improve its expression in corn plants). Alternatively, an expression element may be “synthetic”-meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56 (1), 38-49.

Constitutive promoters include, for example, the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; ALS promoter (U.S. Pat. No. 5,659,026) and the like. Tissue-preferred promoters can be utilized to target enhanced expression within a particular plant tissue. Seed-preferred promoters include both seed-specific promoters active during seed development, as well as seed-germinating promoters active during seed germination. Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Pathogen inducible promoters induced following infection by a pathogen include, but are not limited to those regulating expression of PR proteins, SAR proteins, beta-1,3-glucanase, chitinase, etc. A stress-inducible promoter includes the RD29A promoter (Kasuga et al. (1999) Nature Biotechnol. 17:287-91). One of ordinary skill in the art is familiar with protocols for simulating stress conditions such as drought, osmotic stress, salt stress and temperature stress and for evaluating stress tolerance of plants that have been subjected to simulated or naturally-occurring stress conditions.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, NY: Academic Press), pp. 1-82.

### Morphogenic Factors

A morphogenic factor (gene or protein)—also referred to as a “developmental gene”—may be involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. Introduction of one or more morphogenic factors may improve the frequency or efficiency of transformation or embryogenesis.

In some aspects, the morphogenic factor is a molecule selected from one or more of the following categories: 1) cell cycle stimulatory polynucleotides including plant viral replicase genes such as RepA, cyclins, E2F, prolifera, cdc2 and cdc25; 2) developmental polynucleotides such as Lec1, Kn1 family, WUSCHEL (WUS), Zwille, BBM (babyboom), Aintegumenta (ANT), FUS3, and members of the Knotted family, such as Kn1, STM, OSHI, and SbH1; 3) anti-apoptosis polynucleotides such as CED9, Bcl2, Bcl-X (L), Bcl-W, A1, McL-1, Mac1, Boo, and Bax-inhibitors; 4) hormone polynucleotides such as IPT, TZS, and CKI-1; and 5) silencing constructs targeted against cell cycle repressors, such as Rb, CKI, prohibitin, and weel, or stimulators of apoptosis such as APAF-1, bad, bax, CED-4, and caspase-3, and repressors of plant developmental transitions, such as Pickle and WD polycomb genes including FIE and Medea. The polynucleotides can be silenced by any known method such as antisense, RNA interference, cosuppression, chimerplasty, or transposon insertion. In some aspects, the morphogenic gene is a member of the WUS/WOX gene family (WUS1, WUS2, WUS3, WOX2A, WOX4, WOX5, or WOX9) see U.S. Pat. Nos. 7,348,468 and 7,256,322 and United States Patent Application publications 20170121722 and 20070271628. In some embodiments, the morphogenic gene or protein is a member of the AP2/ERF family of proteins. In some embodiments, the morphogenic factor is a babyboom (BBM) polypeptide, which is a member of the AP2 family of transcription factors.

Introduction of System Components into a Cell

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

For example, the guide polynucleotide (guide RNA, crNucleotide+tracrNucleotide, guide DNA and/or guide RNA-DNA molecule) can be introduced into a cell directly (transiently) as a single stranded or double stranded polynucleotide molecule. The guide RNA (or crRNA+tracrRNA) can also be introduced into a cell indirectly by introducing a recombinant DNA molecule comprising a heterologous nucleic acid fragment encoding the guide RNA (or crRNA+tracrRNA), operably linked to a specific promoter that is capable of transcribing the guide RNA (crRNA+tracrRNA molecules) in said cell. The specific promoter can be, but is not limited to, an RNA polymerase III promoter, which allow for transcription of RNA with precisely defined, unmodified, 5′- and 3′-ends (Ma et al., 2014, Mol. Ther. Nucleic Acids 3: e161; DiCarlo et al., 2013, Nucleic Acids Res. 41:4336-4343; WO2015026887, published 26 Feb. 2015). Any promoter capable of transcribing the guide RNA in a cell can be used and includes a heat shock/heat inducible promoter operably linked to a nucleotide sequence encoding the guide RNA.

The Cas endonuclease, such as the Cas endonuclease described herein, can be introduced into a cell by directly introducing the Cas polypeptide itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock/heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any one of the guided Cas system components can be accompanied by direct delivery (co-delivery) of other mRNAs that can promote the enrichment and/or visualization of cells receiving the guide polynucleotide/Cas endonuclease complex components.

Introducing a guide RNA/Cas endonuclease complex described herein, (representing the cleavage ready cascade described herein) into a cell includes introducing the individual components of said complex either separately or combined into the cell, and either directly (direct delivery as RNA for the guide and protein for the Cas endonuclease and protein subunits, or functional fragments thereof) or via recombination constructs expressing the components (guide RNA, Cas endonuclease, protein subunits, or functional fragments thereof). Introducing a guide RNA/Cas endonuclease complex (RGEN) into a cell includes introducing the guide RNA/Cas endonuclease complex as a ribonucleotide-protein into the cell. The ribonucleotide-protein can be assembled prior to being introduced into the cell as described herein. The components comprising the guide RNA/Cas endonuclease ribonucleotide protein (at least one Cas endonuclease, at least one guide RNA, at least one protein subunit) can be assembled in vitro or assembled by any means known in the art prior to being introduced into a cell (targeted for genome modification as described herein).

Plant cells differ from human and animal cells in that plant cells comprise a plant cell wall which may act as a barrier to the direct delivery of the ribonucleoproteins and/or of the direct delivery of the components.

Direct delivery of a ribonucleoprotein comprising a Cas endonuclease protein and a guide RNA into plant cells may be achieved through particle mediated delivery (particle bombardment. Based on the experiments described herein, a skilled artesian can now envision that any other direct method of delivery, such as but not limiting to, polyethylene glycol (PEG)-mediated transfection to protoplasts, electroporation, cell-penetrating peptides, or mesoporous silica nanoparticle (MSN)-mediated direct protein delivery, can be successfully used for delivering RGEN ribonucleoproteins into plant cells.

Direct delivery of the ribonucleoprotein allows for genome editing at a target site in the genome of a cell which can be followed by rapid degradation of the complex, and only a transient presence of the complex in the cell. This transient presence of the complex may lead to reduced off-target effects. In contrast, delivery of components (guide RNA, Cas9 endonuclease) via plasmid DNA sequences can result in constant expression from these plasmids which in some cases may promote off target cleavage (Cradick, T. J. et al. (2013) Nucleic Acids Res 41:9584-9592; Fu, Y et al. (2014) Nat. Biotechnol. 31:822-826).

Direct delivery can be achieved by combining any one component of the guide RNA/Cas endonuclease complex, representing the cleavage ready cascade described herein, (such as at least one guide RNA, at least one Cas protein, and optionally one additional protein), with a particle delivery matrix comprising a microparticle (such as but not limited to of a gold particle, tungsten particle, and silicon carbide whisker particle) (see also WO2017070032 published 27 Apr. 2017).

In one aspect the guide polynucleotide/Cas endonuclease complex, is a complex wherein the guide RNA and Cas endonuclease protein forming the guide RNA/Cas endonuclease complex are introduced into the cell as RNA and protein, respectively.

In one aspect the guide polynucleotide/Cas endonuclease complex, is a complex wherein the guide RNA and Cas endonuclease protein and the at least one protein subunit of a Cascade forming the guide RNA/Cas endonuclease complex are introduced into the cell as RNA and proteins, respectively.

In one aspect the guide polynucleotide/Cas endonuclease complex, is a complex wherein the guide RNA and Cas endonuclease protein and the at least one protein subunit of a Cascade forming the guide RNA/Cas endonuclease complex (cleavage ready cascade) are preassembled in vitro and introduced into the cell as a ribonucleotide-protein complex. Alternatively, polynucleotides may be introduced into cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

### Cells and Organisms

The presently disclosed polynucleotides and polypeptides can be introduced into a cell. Cells include, but are not limited to, human, non-human, animal, mammalian, bacterial, protist, fungal, insect, yeast, non-conventional yeast, and plant cells, as well as plants and seeds produced by the methods described herein. In some aspects, the cell of the organism is a reproductive cell, a somatic cell, a meiotic cell, a mitotic cell, a stem cell, or a pluripotent stem cell. Any cell from any organism may be used with the compositions and methods described herein, including monocot and dicot plants, and plant elements.

### Animal Cells

The presently disclosed polynucleotides and polypeptides can be introduced into an animal cell. Animal cells can include, but are not limited to: an organism of a phylum including chordates, arthropods, mollusks, annelids, cnidarians, or echinoderms; or an organism of a class including mammals, insects, birds, amphibians, reptiles, or fishes.

The compositions and methods described herein may be used to edit the genome of an animal cell in various ways. In one aspect, it may be desirable to delete one or more nucleotides. In another aspect, it may be desirable to insert one or more nucleotides. In one aspect, it may be desirable to replace one or more nucleotides. In another aspect, it may be desirable to modify one or more nucleotides via a covalent or non-covalent interaction with another atom or molecule. Genome modification may be used to effect a genotypic and/or phenotypic change on the target organism. Such a change is preferably related to an improved phenotype of interest or a physiologically-important characteristic, the correction of an endogenous defect, or the expression of some type of expression marker. In some aspects, the phenotype of interest or physiologically-important characteristic is related to the overall health, fitness, or fertility of the animal, the ecological fitness of the animal, or the relationship or interaction of the animal with other organisms in its environment.

Cells that have been genetically modified using the compositions or methods described herein may be transplanted to a subject for purposes such as gene therapy, e.g. to treat a disease, or as an antiviral, antipathogenic, or anticancer therapeutic, for the production of genetically modified organisms in agriculture, or for biological research.

### Plant Cells and Plants

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to: oilseed rape or Canola) (Brassica napus, B. campestris, Brassica rapa, Brassica. juncea), alfalfa (Medicago sativa),), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum.

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization. The present disclosure finds use in the breeding of plants comprising one or more introduced traits, or edited genomes.

In an embodiment, crossover rates or crossover frequency are increased. In an embodiment, crossover distribution is improved in genomic regions that are not active for recombination. Targeted introduction of double strand breaks (DSBs) during meiosis induce homologous recombination in crop plants, where a large chromosomal fragment is recombined compared to a control crop plant. In an embodiment, DNA-binding capability of Cas endonucleases (e.g., dCas9) to guide factors responsible for double strand breaks (i.e., not a programmable nuclease mediated break) increase crossover frequency and/or distributions.

A non-limiting example of how two traits can be stacked into the genome at a genetic distance of, for example, 5 CM from each other is described as follows: A first plant comprising a first transgenic target site integrated into a first DSB target site within the genomic window and not having the first genomic locus of interest is crossed to a second transgenic plant, comprising a genomic locus of interest at a different genomic insertion site within the genomic window and the second plant does not comprise the first transgenic target site. About 5% of the plant progeny from this cross will have both the first transgenic target site integrated into a first DSB target site and the first genomic locus of interest integrated at different genomic insertion sites within the genomic window. Progeny plants having both sites in the defined genomic window can be further crossed with a third transgenic plant comprising a second transgenic target site integrated into a second DSB target site and/or a second genomic locus of interest within the defined genomic window and lacking the first transgenic target site and the first genomic locus of interest. Progeny are then selected having the first transgenic target site, the first genomic locus of interest and the second genomic locus of interest integrated at different genomic insertion sites within the genomic window. Such methods can be used to produce a transgenic plant comprising a complex trait locus having at least 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31 or more transgenic target sites integrated into DSB target sites and/or genomic loci of interest integrated at different sites within the genomic window. In such a manner, various complex trait loci can be generated.

In certain embodiments, methods and compositions provided herein enable NHEJ-mediated repair and further enable somatic homologous recombination events. These events include gene conversions and crossovers. Use of CRISPR-Cas machinery to induce targeted DSB influence recombination between homologous chromosomes in crop plants.

In another embodiment, use of hybrids of crop plants from genetically distinct accessions that contain heterozygous mutations in a gene of interest (or SNPs in a QTL of interest) are obtained through homologous recombination-mediated somatic recombination.

In certain embodiments, recombination between homologous chromosomes by NHEJ might is preferred to homologous recombination-mediated recombination. For example, simultaneous DSB induction in both homologous chromosomes increases reciprocal chromosomal exchange of genetic material by NHEJ.

Double strand breaks (DSBs) in chromosomal DNA are usually repaired restoring the original order of the chromatin using either non-homologous end joining (NHEJ) or homology directed repair (HDR) pathways. As multiple DSBs can occur simultaneously, in some cases their repair may result in large chromosomal aberrations such as deletions, duplications, and inversions (if two breaks occurred in the same chromosome), or in reciprocal or non-reciprocal translocations and chromosomal fusions involving fragments of different chromosomes. Although infrequent, these types of random chromosomal rearrangements are known to occur spontaneously and considered important elements of plant adaptation and speciation. However, targeted or induced DSBs in chromosomal do not occur in nature or spontaneously, or at a frequency that is practical.

The ability to discover or detect large-scale chromosomal rearrangements between maize inbred lines has previously been hampered by several factors, such as the reliance on reference-based approaches to build new reference genomes and the need for genetic markers to place contigs/scaffolds into pseudomolecules. In addition, the lack of a robust high-throughput process for generating ab initio reference genomes made it unfeasible to characterize many lines for the presence of large chromosomal rearrangements. High-quality chromosome-scale genome assemblies for crop plants such as maize are used herein to elucidate and map large-scale chromosomal rearrangements, differentiating them from mis-assembly. Chromosomal rearrangements are spontaneous and unpredictable and therefore, cannot be reliably used in breeding programs.

Linkage drag generally refers to yield reduction (or the display of an unfavorable phenotype of interest) is caused by a less than ideal genetic exchange in between the respective traits. Therefore, traits in linkage disequilibrium or traits that reside within a tight linkage region on the same chromosome are, generally, genetically linked. The targeted induction of crossovers and inversions enables these linkages to be exploited or disrupted. Heterologous, induced crossovers between homologous chromosomes can break the linkage of traits linked by close physical proximity or otherwise in linkage disequilibrium. Alternatively, induced, artificial crossovers are used to establish new linkage groups by combining traits on the same chromosome to be in close chromosomal proximity. Heterologous induced inversions substantially reduce regions from genetic exchange by promoting linkage groups. Both chromosomal rearrangements pave the way to controlling and manipulating the natural recombination landscape in a targeted manner.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention. The examples are offered for illustrative purposes and are not intended to limit the scope of the invention in any way. Efforts have been made to ensure accuracy with respect to numbers used (e.g., amounts, temperatures, etc.), but some experimental error and deviation should, of course, be allowed for.

### Example 1: Particle Bombardment Transformation of Corn Plants

In this example, transformation of immature maize embryos via particle bombardment is described. It is understood that a similar protocol may be used for the transformation of other plants, such as (but not limited to): soybean, cotton, canola, wheat, rice, sorghum, or sunflower. Prior to bombardment, 10-12 DAP immature embryos are isolated from ears of a corn plant and placed on culture medium plus 16% sucrose for three hours to plasmolyze the scutellar cells. Single plasmids or multiple plasmids may be used for each particle bombardment. In one example, plasmids include: 1) a plasmid comprising a donor cassette, 2) a plasmid comprising an expression cassette for the double-strand-break-inducing agent, and 3) a plasmid comprising an expression cassette for the morphogenic factor.

To attach the DNA to 0.6 μm gold particles, the plasmids are mixed by adding 10 μl of each plasmid together in a low-binding microfuge tube (Sorenson Bioscience 39640T). To this suspension, 50 μl of 0.6 μm gold particles (30 μg/μl) and 1.0 μl of Transit 20/20 (Cat No MIR5404, Mirus Bio LLC) are added, and the suspension is placed on a rotary shaker for 10 minutes. The suspension is centrifuged at 10,000 RPM (˜9400×g) and the supernatant is discarded. The gold particles are re-suspended in 120 μl of 100% ethanol, briefly sonicated at low power and 10 μl is pipetted onto each carrier disc. The carrier discs are then air-dried to remove all the remaining ethanol. Particle bombardment is performed using a Biolistics PDF-1000, at 28 inches of Mercury using a 425 PSI rupture disc. After particle bombardment, the immature embryos are selected on 506J medium modified to contain 12.5 g/l mannose and 5 g/l maltose and no sucrose. After 10-12 weeks on selection, plantlets are regenerated and analyzed using qPCR.

Components may be delivered as DNA, RNA, protein, or any combination of the preceding. In one example, Cas9 and the guide RNA were delivered as a ribonucleoprotein (RNP) complex. Previously, a method for Cas9-gRNA delivery in the form of ribonucleoproteins (RNPs) using gold microparticles was described. A method for activation of broken pre-integrated selectable marker gene through non-homologous end joining (NHEJ) mechanism upon Cas9-gRNA delivery as DNA vectors or RNP complex (WO2017/070029 A1). Similarly, interaction between two regions/loci located on two different chromosomes is generally limited and frequencies of chromosomal rearrangements between chromosomes are low.

### Example 2: Agrobacterium-Mediated Transformation of Corn Plants

In this example, transformation of immature maize embryos via Agrobacterium mediation is described. It is understood that a similar protocol may be used for the transformation of other plants, such as (but not limited to): soybean, cotton, canola, wheat, rice, sorghum, or sunflower.

### Preparation of Agrobacterium Master Plate.

Agrobacterium tumefaciens harboring a binary donor vector is streaked out from a −80° C. frozen aliquot onto solid 12V medium and cultured at 28° C. in the dark for 2-3 days to make a master plate.

### Growing Agrobacterium on Solid Medium.

A single colony or multiple colonies of Agrobacterium are picked from the master plate and streaked onto a second plate containing 8101 medium and incubated at 28° C. in the dark overnight.

Agrobacterium infection medium (700 medium A; 5 ml) is added to a 14 mL conical tube in a hood. About 3 full loops of Agrobacterium from the second plate are suspended in the tube and the tube was then vortexed to make an even suspension. One mL is transferred to a spectrophotometer tube and the optical density (550 nm) of the suspension is adjusted to a reading of about 0.35-1.0. The Agrobacterium concentration is approximately 0.5 to 2.0×10⁹ cfu/mL. The final Agrobacterium suspension is aliquoted into 2 mL microcentrifuge tubes, each containing about 1 mL of the suspension. The suspensions are then used as soon as possible.

### Growing Agrobacterium on Liquid Medium.

Alternatively, Agrobacterium can be prepared for transformation by growing in liquid medium. One day before infection, a 125 ml flask is prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 uL spectinomycin (50 mg/mL). A half loopful of Agrobacterium from a second plate is suspended into the flasks and placed on an orbital shaker set at 200 rpm and incubated at the 28° C. overnight. The Agrobacterium culture is centrifuged at 5000 rpm for 10 min. The supernatant is removed and the Agrobacterium infection medium was added. The bacteria are resuspended by vortex and the optical density (550 nm) of Agrobacterium suspension is adjusted to a reading of about 0.35 to 2.0.

### Maize Transformation

Ears of a maize (Zea mays L.) cultivar are surface-sterilized for 15-20 min in 20% (v/v) bleach (5.25% sodium hypochlorite) plus 1 drop of Tween 20 followed by 3 washes in sterile water. Immature embryos (IEs) are isolated from ears and are placed in 2 ml of the Agrobacterium infection medium. The optimal size of the embryos varies based on the inbred, but for transformation with WUS2 and ODP2 a wide size range of immature embryo sizes could be used. The solution is drawn off and 1 ml of Agrobacterium suspension was added to the embryos and the tube vortexed for 5-10 sec. The microfuge tube is allowed to stand for 5 min in the hood. The suspension of Agrobacterium and embryos are poured onto 7101 (or 562V) co-cultivation medium (see Table 2). Any embryos left in the tube are transferred to the plate using a sterile spatula. The Agrobacterium suspension is drawn off and the embryos placed axis side down on the media. The plate is sealed with Parafilm M® film (moisture resistant flexible plastic, available at Bemis Company, Inc., 1 Neenah Center 4th floor, PO Box 669, Neenah, WI 54957) and incubated in the dark at 21° C. for 1-3 days of co-cultivation.

Embryos are transferred to resting medium (605T medium) without selection. Three to 7 days later, they are transferred to maturation medium (289Q medium) supplemented with a selective agent

### Example 3: Morphogenic Factors Improve Transformation Frequency, Pericentric and Paracentric Chromosomal Inversions

Morphogenic factor genes (also referred to as “developmental genes”, for example but not limited to: ODP2 and WUS) are desired components of the transformation process: their delivery into plant cells facilitates cell division and significantly increases transformation frequencies. Moreover, these genes allow successful transformation of many elite genotypes or elite crop germplasm, which transformation, otherwise, cannot be effectively accomplished. Parameters of the transformation protocol can be modified to ensure that the BBM activity is transient. One such method involves precipitating the BBM-containing plasmid in a manner that allows for transcription and expression, but precludes subsequent release of the DNA, for example, by using the chemical PEI.

In this manner, PEI-precipitation can be used to deliver transient expression of BBM and/or WUS2. For example, the particles are first coated with UBI::BBM::pinII using PEI, then coated with UBI::moPAT˜YFP using a water-soluble cationic lipid transfection reagent, and then bombarded into scutellar cells on the surface of immature embryos. PEI-mediated precipitation results in a high frequency of transiently expressing cells on the surface of the immature embryo and extremely low frequencies of recovery of stable transformants. Thus, it is expected that the PEI-precipitated BBM cassette expresses transiently and stimulates a burst of embryogenic growth on the bombarded surface of the tissue (i.e. the scutellar surface), without stable genomic integration. The PAT˜GFP plasmid released from the Ca++/gold particles is expected to integrate and express the selectable marker at a frequency that results in substantially improved recovery of transgenic events. As a control treatment, PEI-precipitated particles containing a UBI::GUS::pinII (instead of BBM) are mixed with the PAT˜GFP/Ca++ particles. Immature embryos from both treatments are moved onto culture medium containing 3 mg/l bialaphos. After 6-8 weeks, it is expected that GFP+, bialaphos-resistant calli is observed in the PEI/BBM treatment at a much higher frequency relative to the control treatment (PEI/GUS).

As an alternative method, the BBM plasmid is precipitated onto gold particles with PEI, and then introduced into scutellar cells on the surface of immature embryos, and subsequent transient expression of the BBM gene elicits a rapid proliferation of embryogenic growth. During this period of induced growth, the explants are treated with Agrobacterium using standard methods for maize (see Example 1), with T-DNA delivery into the cell introducing a transgenic expression cassette such as UBI::moPAT˜GFPm::pinII. After co-cultivation, explants are allowed to recover on normal culture medium, and then are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS). It may be desirable to “kick start” callus growth by transiently expressing the BBM and/or WUS2 polynucleotide products. This can be done by delivering BBM and WUS2 5′-capped polyadenylated RNA, expression cassettes containing BBM and WUS2 DNA, or BBM and/or WUS2 proteins.

Use of morphogenic factors, e.g., BBM and/or WUS, had several advantages for pericentric and paracentric chromosomal inversions. First, some genotypes are recalcitrant to some types of transformation. Second, forcing cells into division increases the chances of successful outcome, as it activates DSB repair mechanisms. Third, chromosomes positioning in the nucleus is not random but occupy specific territories specific to both cell type and tissue type. Moreover, chromatin organization and interaction are different and depend on specific cell cycle stages. For example, G1 stage is transcriptionally active and characterized by a short range intrachromosomal contact compared to long-range contacts. By association of Hi-C data with cell cycle progression, the prevalence of local contacts during interphase and the enrichment of long-range mitotic contacts during mitosis and early G1 stages was affirmed. Therefore, expression of morphogenic genes, which pushes cell to replication (S-phase) and to cell division likely increases chances of “improper” DSB repair and generation of the desired chromosomal rearrangements. Other cell cycle proteins and transcription factors may also contribute to the successful outcome of pericentric and paracentric chromosomal inversions.

### Example 4: Increased Chromosomal Pericentric and Paracentric Inversions Frequency in Plant Cells Through Oligonucleotides

In most experiments, chromosomal rearrangements (deletions, duplications, inversions, and translocations) result from a random ‘improper’ DSB repair, therefore, happen with very low frequencies. Further optimization and frequency improvement of the desired pericentric and paracentric chromosomal inversions can be accomplished by various approaches and modifications in the experimental designs described below.

Oligonucleotides with Sequence Homology to DSB Ends

Co-delivery of DSB reagents and ‘translocation’ single-stranded oligonucleotides (ssONDs) with homology to chromosomal ends at two different DSBs can guide ‘improper’ DNA repair and increase frequency of translocation-derived chromosomes in cells up to 10-fold.

This approach is used in plant cells to direct DSB repair with favorable outcomes to generate not only translocations, but also other types of chromosomal rearrangements such as inversions, deletions, and duplications. In certain embodiments, this approach is feasible with particle gun-mediated transformation allowing co-delivery of DSB reagents (e.g., CRISPR-Cas nucleases) in the form of DNA plasmid(s) or ribonucleoprotein (RNP) complex, selectable marker gene, and varying amounts of ss ONDs. This approach can also be used for chromosome engineering in plant cells after delivery of editing reagents by transfection or electroporation, in the case of plant protoplasts (i.e., lacking a cell wall).

In certain other embodiments, to drive DSB repair with the desirable chromosome rearrangement(s), double-stranded oligonucleotides (dsONDs) with homologies to chromosomal ends at two different DSBs can also be used. This approach is based on Homology Directed Repair (HDR) of DSBs in the presence of a linking donor template. HDR-based gene insertion with frequencies as high as 10-15% have been demonstrated in our recent experiments indicating that HDR can be very efficient in actively dividing plant cells.

dsONDs can be either transfected/electroporated to plant protoplasts or delivered to intact plant cells with cell walls using particle bombardment. In these cases, dsODNs can be co-delivered with DSB reagents in the form of ribonucleoprotein (RNP) complexes or DNA plasmid(s) and morphogenic and selectable marker genes. Alternatively, the oligonucleotide sequences can be cloned into a plasmid vector and flanked with the target sequences with PAM. In certain embodiments, dsODNs can be then released from the plasmid DNA upon the target sites cleavage by a Cas nuclease used in the experiment.

In case of Agrobacterium-mediated delivery, oligonucleotides can be a part of a T-DNA vector and flanked with a site-directed nuclease target sites with PAM to release it from the T-DNA upon cleavage. In addition, to increase the copy number of the dsONDs and the frequency of the desired chromosomal rearrangements, multiple copies of the oligonucleotides can be present in the T-DNA as a concatemer, where each copy is separated by the nuclease target sites with PAM. Viral vectors capable of providing several copies of the oligonucleotides having similarity to the target sites are also suitable for use in this Example, based on the guidance provided for by other methods demonstrated herein.

### Example 5: Selection of Chromosomal Target Sites in Plant Cells for Pericentric and Paracentric Chromosomal Inversions

In case of deletions, inversions, duplications and non-homologous chromosome translocations, selected target sites are preferred to be i) specific, ii) located in non-coding regions, and iii) not in a highly repetitive sequence region. Low recombinogenic regions in the pericentromeric regions of the chromosomes are selected for targeted DNA breaks or nicks. Similarly, chromosomal regions in the recombinogenic subtelomeric regions are selected for targeted DNA breaks. Introducing such targeted breaks increase the chance of pericentric and paracentric inversions. In certain embodiments, the targeted DNA breaks occur in the same arm of the chromosome.

In case of translocations between homologous and homeologous chromosomes with high level of sequence identity, target sites should contain enough polymorphism between the two chromosomes providing chromosome specific DSBs. In addition, to allow using ssOND or dsONDs, target sites are selected-50-100 or more nucleotides away from each other to provide very specific sequences for oligos design and further molecular analysis of the translocation events.

### Example 6: Chromosomal Pericentric and Paracentric Inversions in Plant Cells Improve Breeding Methods and Increase Recombination Frequency in Plants

Chromosomal inversions (e.g., pericentric and paracentric) suppress recombination between chromosomes with different orientations of the inverted regions. There are multiple examples of chromosomal inversions existing in crop species, including maize, soy, canola, sunflower, and in their undomesticated relatives. Genetic recombination allows a breeder to select for genetic gain because it creates new combinations of alleles at genetically linked loci. For example, one line has a favorable allele at first locus, and an unfavorable allele at a second, and the other line has an unfavorable allele at the first locus and a favorable allele at the second, and the alleles are genetically linked on the same chromosome or linkage group. Then, crossing the two lines allows the breeder to select offspring of the F1 carrying the favorable alleles at both loci as the result of genetic recombination between the chromosomes of the parents. The case in which unfavorable and favorable alleles reside on the same chromosome is called repulsion phase linkage, and the case where the favorable alleles reside on the same chromosome is called coupling phase linkage. Thus, chromosomal inversions suppress recombination and reduce genetic gain through conversion of repulsion phase linkage to coupling phase linkage.

Meiotic recombination can be restored in hybrids that contain an allele or a genetic segment in a heterozygous state. Crossover rates are be restored in a hybrid background. Reversing evolutionary-derived chromosomal rearrangements help expose new genetic material within the chromosomal rearrangement for meiotic recombination. CRISPR-Cas-mediated inversion in a crop plant was demonstrated herein.

The reorientation of an inverted chromosomal region back to, for example, the ancestral orientation, allows recombination between alleles that otherwise would be less likely. Such chromosomal reorientation, that may spontaneously occur, may occur at extremely low frequencies, thus making it impractical for the selection of new lines and varieties in breeding. Therefore, CRISPR-Cas facilitated inversions (also non-natural, induced reversions or inversions) and large chromosomal fragment reorientation with higher frequencies, that can enable a breeder to select for favorable combinations of alleles that would otherwise be practically unfeasible in a breeding program.

In other cases, it may be desirable to invert a portion of a chromosome to “lock down” a particular locus and prevent its recombination, thereby maintaining linkage of the desirable traits for breeding purposes.

When DSBs are generated on each side of the inversion, the repair can be incorrect and result in intrachromosomal translocation. This outcome results in the re-orientation of the pericentric inversion. To test feasibility of a large chromosomal inversion facilitated by CRISPR-Cas9 technology, we performed reorientation (reversion) of a 76 Megabase (Mb) long pericentric inversion that naturally occurred in chromosome 2 of one genotype of maize. Six target sites (three on each side of the inversion) were identified and the corresponding gRNAs were tested transiently for mutation frequencies in each site. Two target sites with highest mutation frequencies (one on each side of the inversion) were selected and used in the transformation experiment.

Feasibility of large chromosomal inversions using site-specific double strand break (DSB) technology with practical frequencies and thereby enabling chromosomal rearrangements of varying sizes to suit a breeder's need in a variety of crop plants. For example, centromeric regions in maize contain large numbers of genes and QTLs within regions of small genetic map length due to low recombination rates. It is feasible that different QTLs with favorable or unfavorable alleles are linked in repulsion in regions of less than 1 centimorgan. Developing this example, a breeder could wish to improve both yield and disease resistance in a population where the favorable yield QTL allele I (yield+) s linked to an uunfavorable disease resistance allele (disease−), with a 1% chance of recombination between them. To achieve the desired arrangement with favorable alleles for both QTL (yield+, disease+), a breeder would cross the yield+, disease− individual with another line with the opposite arrangement, yield−, disease+. Recombination in the F1 would generate F2 or doubled haploid progeny with the favorable arrangement of yield+, disease+ at a rate of 1%. Generating a few hundreds of progeny would effectively guarantee observation of favorable recombinations. If, however, if the genes lie at opposing sides of a pericentromeric (encompassing the centromere) inversion in one of the two parental arrangements, recombination would be drastically reduced. Recombination between opposing orientations of an inversion encompassing the centromere typically occurs only by double crossover, otherwise lethality of the gametes can result. These double crossovers can be inhibited by recombination interference, but even assuming no interference the double crossover rate in this example is expected be on the order of one in ten thousand progeny as opposed to one in one hundred. Generating the number of progeny needed to observe the favorable allele in this case is beyond standard breeding practices. For example, disease resistance alleles that normally do not co-segregate with, for example, yield QTLs. After chromosomal engineering, these QTLs are now arranged in a targeted fashion in a chromosome such that they are in linkage disequilibrium or co-segregates more often than what would be observed for a control breeding population. This engineered co-segregation of favorable alleles (for example uncoupled from unfavorable alleles), QTLs, SNPs help drive genetic gain and reduce breeding cycle time to achieve those results.

### Example 7: Targeted Stable Chromosomal Centromeric Inversions in Plant Cells to Preserve Linkage Disequilibrium and Reduce Recombination Frequency within a Set of Favorable Alleles in Plants

When coupling phase linkage exists, either with native QTL or by insertion of multiple transgenes into the same chromosomal region and no additional recombination within that desired genomic locus is desired, an inversion or translocation of that locus through a targeted approach can reduce the rate of recombination between the loci, thus maintaining the desired state. For example, after the desired panel of alleles, QTLs, SNPs, transgenes, CRISPR-edited variants, are assembled in a region of the chromosome through conventional breeding or other facilitated breeding methods or other site-directed insertion methods, that region or a segment of the chromosome is inverted to suppress recombination between the desired alleles or with those alleles that may perturb the desired linkage state. Conversely, inducing targeted inversions of a chromosomal region of a crop plant (i.e., creating a novel chromosomal inversion within a chromosomal region) is used to stabilize genetic linkages between desirable traits. These induced heterologous, non-natural, chromosomal inversions make that segment of the chromosomal region inaccessible for recombination between the homologous chromosomes during meiosis, thereby stabilizing or fixing favorable alleles in genomic window.

For example, if the region containing these alleles is 10 centimorgans in genetic length, one in ten progeny of crosses with an individual not carrying the arrangement would possess a crossover yielding only some of the desired alleles being maintained. If the inversion suppresses recombination except by double-crossover, an inversion of the desired allele arrangement could reduce the number or progeny carrying undesirable genotypes by approximately ten-fold from 10% to 1%. This should be viewed as a maximum recombination rate, as interference could lead to an even smaller observed crossover rate. If the favorable allelic region was not inverted but instead translocated to a different chromosome, especially a centromeric region with low recombination rates, the rearrangement could effectively eliminate recombination among the favorable alleles. This would enable the favorable arrangement is maintained in all cross progeny and eliminate the need for laborious screening by sequencing or genetic markers to confirm the arrangement. rearranged or inverted to a different chromosome or different part of the same chromosome.

### Example 8: Large Chromosomal Peri and Paracentric Inversions in Plant Cells to Link Distant Chromosomal Segments in Plants

In an embodiment, the favorable allele of a QTL is for example, characterized by an interval on a genetic or a physical map that contains hundreds, thousands, or millions of bases and multiple genes. It is often advantageous to narrow down the QTL interval and identify the specific genes, gene regulatory regions and DNA sequence changes that cause the differences in phenotype between lines carrying favorable and unfavorable alleles. This approach enables more efficient and accurate selection of the favorable alleles by using genetic markers that are more tightly linked with the causative DNA sequence polymorphisms.

In another embodiment, narrowing of the QTL interval also enables introgression of the causative polymorphisms into a new line or variety, through a smaller genomic region or through smaller genomic intervals within a segment of a chromosome. This genomic locus allows to reduce linkage drag—the introgression of unfavorable alleles for other traits that might otherwise be linked to the favorable QTL region. Traditionally, narrowing of a QTL interval to identify causative SNPs, genetic segments, has been done by fine-mapping, which includes using genetic crosses between individuals or populations carrying different QTL alleles to generate genetic recombination in the QTL region. Genetic markers can then be used to identify where and in which individuals, recombinations have occurred and relate these data to the phenotypic performance of the offspring carrying each recombination. As the genetic map interval of the QTL region narrows, recombination in the target region become substantially rare, which are needed to help further refine the region. Therefore, the efficiency and feasibility of fine mapping reduces as the QTL region is refined. In some cases, a genetic map interval that is sufficiently narrow to reduce recombination frequencies to less than 1% can still contain millions of DNA base pairs and hundreds of genes. A region of this physical size will often not be sufficiently small for the desired application or may still contain deleterious alleles that are harder to breed out because of the tight linkage.

Series of deletions of selected, various size DNA fragments using CRISPR-Cas technology can help to reveal the minimal causative region within the QTL. For example, if there is a functional gene conferring disease resistance vs. the absence of a functional allele, then deletion of the region containing the gene would reveal a difference in phenotype, whereas deletion of the unfavorable, non-functional allele, would show no difference in phenotype. For a QTL interval with many genes, it can be extremely beneficial if large deletions (from kilobases to several megabases) are used to identify DNA region with a causative difference. Then this region could be further analyzed by smaller deletions until a sufficiently small genetic region is defined. Thus, generation of CRISPR-Cas induced large deletion lines substantially reduces the cost and increase the efficiency of narrowing QTL regions over traditional fine mapping that may need a substantially large number of lines and in many cases may not be practical especially if the recombination frequency is substantially low. Fine mapping traditionally relies on crossovers generated during meiosis, which can require screening large numbers of progeny with genetic markers. Since fine-mapping often focuses on small genomic regions less than 1 centimorgan in length, hundreds or even thousands of progeny must be created and genotyped with genetic markers spanning the region of interest to first detect recombination, followed by additional marker screening to define the specific recombination breakpoint. In some cases, this requires the creation of new genetic markers for screening and multiple generations of recombination to generate the desired panel of recombinant lines.

With very small regions of the genome containing only a few genes, targeted CRISPR-cas gene deletions could validate which genes contribute to the QTL more quickly than fine mapping. Since the deletions are targeted, their expected breakpoints are known in advance. This enables screening of the breakpoints to be done with a pre-defined set of markers. However, although the genomic region of interest for many QTL is small in terms of the genetic map, it can still be quite large in terms of physical DNA bases. In the low recombination regions of the maize genome such as the pericentromeric regions, a 1 cM interval can contain hundreds of thousands to tens of millions of base pairs and tends to hundreds of genes. The number of small deletions needed to validate whether a gene contributes to a QTL would be impractical in this case. Large CRISPR-cas induced deletions can generate a deletion panel in parallel which narrows down the region of interest by ruling out many genes at a time with each deletion. For example, a QTL region of 100 genes could be deleted with 5 large deletions of 20 genes. For a single-gene QTL only one of these deletions would validate as impacting the trait, thus narrowing the search space from 100 to 20 genes. A small number of additional deletions could further narrow the search space until the gene of interest is identified.

One challenge with large deletions is that a homozygous deletion of multiple genes will often create negative or lethal effects that obscure the phenotype of interest or render it difficult to measure. There is also the chance of deleting a gene that is important for the molecular pathways contributing to the phenotype of interest even if the gene does not contain any genetic variation contributing to the QTL, thus generating a false positive. These challenges could be overcome by testing the large deletion can be tested in a hemizygous state, in which a line or variety carries one intact allele and one deleted allele. If the deleted allele is the unfavorable allele and the favorable allele is intact, then the line will show a more favorable phenotype than an isogenic line carrying an intact unfavorable allele and a deleted favorable allele. These reciprocally hemizygous lines can be generated by crossing a line carrying a homozygous deletion of one allele with a line carrying an intact version of the other allele. Plants carrying such hemizygous states could be generated by identifying transformed embryos for which the targeted region was deleted on only one of the two diploid chromosomes.

CRISPR-Cas technology can be successfully used to generate chromosomal inversions of variable sizes ranging from several kilobases to several megabases to reduce QTL interval and thereby enabling generating chromosomal segments that contain the causative genetic elements and reduced/minimal deleterious DNA.

### Example 9: Chromosomal Centromeric Inversions in Plant Cells Improve Trait Introgressions in Breeding

Introgression of individual genes or QTLs that provide diversity or a specific desired trait such as disease or stress tolerance is an imperative tool for plant breeding. As described above, linkage drag, which refers to the effects of genes physically linked to the desired genes or QTLs, is usually undesirable, especially if the genes or QTLs are coming from germplasm that is not elite or adapted to the target environment. Traditionally, desired traits would be introgressed into elite germplasm by multiple generations of backcrossing. Backcrossing alone is not usually successful without the use of molecular markers to identify any remaining undesired genetic elements from the donor parent. Traditional backcrossing methods on average would take 6 or more generations and still may not fully recover the elite genotype in the linked genetic regions. Gene or QTL relocation using CRISPR-Cas technology coupled with CRISPR-Cas assisted fine mapping described above, could save more than two years of time compared to a traditional backcrossing strategy and would eliminate the concern for any remaining linkage drag or undesired genes in the rest of the genome.

Although linkage drag is commonly suggested as the source of subpar performance compared to the elite parent, it is possible that the introgressed gene itself could have detrimental effects (if the negative effect is due to linkage drag or to the gene itself) on the plant, therefore, causing issues with yield or other agronomic characteristics. This specific translocation would allow confirmation if the gene itself has a detrimental effect. In more traditional breeding systems, many generations of backcross would be required, assuming it is linkage drag, but it would not be confirmed until all surrounding sequence is removed.

If a desirable trait is associated with a specific and defined gene, it can be cloned into a plasmid vector, flanked with regions of homology and inserted into the desirable location of elite genotype through HDR approach. However, if trait is associated with a QTL that may span hundreds of kilobases, cloning and subsequent introduction into the genome often can be impractical. Here application of targeted DNA break for relocation of a QTL of a large size into any desirable location in a crop plant species through chromosomal centromeric inversion is described.

### Example 10: Targeted Modifications in Plant Cells to Increase Chromosomal Centromeric Inversions

Further optimization and frequency improvement of the desired chromosomal centromeric inversions can be accomplished by various approaches and modifications in the experimental designs described below.

### Extending Time of DSB

To increase frequency of desired chromosomal rearrangement(s), the recurrent cleavage at the intended site may be beneficial. Recurrent cutting can be accomplished by using Cas nucleases with cleavage outside of the target site (e.g. Cpf1). Alternatively, it can also be accomplished applying recurrent cutting technology. In addition, similar result can be obtained when two gRNAs targeting sites within close vicinity from each other (up to 30-40 nucleotides) are used. In this case, first ribonucleoprotein complex binds and cleaves the first site making the second site not accessible (physically blocking). If the first DSB is repaired with indel, the second site can be cleaved generating a new DSB expanding window of opportunity for the desired rearrangement to take place. Moreover, in combination with morphogenic genes (BBM and WUS) facilitating cell division, the recurrent cutting approach may be beneficial as it potentially allows generation of the second DSB at a different stage of cell cycle (S or G2 phase) when inter-chromosomal interaction is more feasible.

### PAM Orientation to Reduce NHEJ and Improve Chromosomal Rearrangements

As Cas9 protein remains associated with one end of the DSB, the one with PAM, potentially for several hours after target site cleavage, it protects that end from degradation but also from NHEJ components (e.g. Ku70/80) and provides protection/prevention of proper repair of the DSB.

Therefore, by selection of target sites and their orientation, ‘improper’ repair events can be increased and desired outcome (good for all types of rearrangements-deletions, translocations, and inversions) is obtained. For example, frequency of deletion of a chromosomal fragment is increased by designing target sites with PAM sequencing looking inside inverse the fragment to be deleted. In this case, not protected chromosomal ends will be likely rejoined through NHEJ repair pathway resulting in deletion. Alternatively, DSBs in two different chromosomes can be ‘improperly’ repaired resulting in inter-chromosomal translocation.

Oligonucleotides with Sequence Homology to DSB Ends

It has been demonstrated that co-delivery of DSB reagents and ‘translocation’ single-stranded oligonucleotides (ss ONDs) with homology to chromosomal ends at two different DSBs can guide ‘improper’ DNA repair and increase frequency of translocation-derived chromosomes can be used in plant cells to direct DSB repair with favorable outcome to generate inter-chromosomal translocation(s). This approach is especially feasible with particle gun-mediated transformation allowing co-delivery of DSB reagents in the form of DNA plasmid(s) or ribonucleoprotein (RNP) complex, selectable marker gene, and high amount of ss ONDs.

### Cell Cycle-Specific Expression of Cas9 Nuclease

As described above, chromatin organization and interaction are different in different cell cycle stages. Thus, ‘improper’ DSB repair with desired chromosomal rearrangements may occur with different frequencies depending on the cell cycle stage. Therefore, expression of a Cas nuclease and generation of targeted DSBs during S-phase may further increase chances of desired chromosomal rearrangements. Cell cycle stage-specific expression of Cas nuclease can be accomplished by using, for example, S-phase-specific promoter like ZM-RNR2A. Ribonucleotide Reductase (RNR) is an essential enzyme for de novo synthesis of deoxyribonucleotides, which expression is limited to S-phase.

### Modulating Chromosome Orientation to Enhance Desired Chromosomal Rearrangements

Physical proximity of chromosomal DSB ends will increase chances of their re-joining through NHEJ repair pathway. Therefore, bringing together chromosomal fragments of interest may increase frequencies of chromosomal rearrangement with desired outcomes. This statement is true for any type of chromosomal rearrangements including deletions, inversions, and translocations.

Such physical proximity of desired chromosomal ends in crop plant genomes can be accomplished, for example, by utilization of Cas proteins fused to a unique, reversible chemically induced system that uses phytohormone S-( [symbol] )-abscisic acid (ABA) and modified components of the plant ABA signaling pathway described by Morgan et al. (2017) (Nat Comm 8:15993; DOI: 10.1038/ncomms15993). First, Cas nucleases are directed to their target loci by the corresponding gRNAs. After cleavage, Cas9 nucleases remain associated with PAM side of the DSB. As the result, chromosomal ends desired to be repaired are brought to proximity after addition of ABA, which facilitates dimerization of PYL1 and ABI1 domains fused to Cas nucleases. Furthermore, other dimerization systems, such as rapamycin induced interaction between FKBP12 and FRAP, or FKCsA induced interaction between FKBP12 and cyclophilin, can also be used.

Alternatively, two dead Cas (dCas) orthologous nucleases different from the one used to generate DSB, can be fused to dimerizing components described above. These dCas nucleases bind to the corresponding target sites and juxtaposition of the two chromosomal ends is accomplished by induction of the dimerizing components.

### Large Fragment Centromeric Inversion and Gene Stacking

In one aspect, two consecutive translocations are performed: first translocation between non-homologous chromosomes to bring the QTL to the desired location; second-restoring the telomere of the chromosome with CTL. In one aspect, a region of homology is introduced into the intended TS. In one aspect, fusions with two Cas9 orthologs (one Cas9 for the target site and the second one for releasing fragment to be relocated) are used, that can make them form dimers, bring them close to a locus.

### Example 11: Centromeric Inversions of Chromosomal Regions Associated with Disease in Plant Cells

In this example, chromosomal region or pericentromeric segments, including QTLs associated with one or more diseases in crop plants such as corn, soybean, cotton, canola, wheat, rice, sorghum, or sunflower are rearranged (e.g., inversion, translocation) such that those chromosomal regions are in a preferred chromosomal configuration that enables faster trait introgression, reduced linkage drag, optimal linkage disequilibrium compared to control and other breeding enhancements. In an embodiment, disease chromosomal segment is translocated to a preexisting transgenic locus containing one or more insect and/or herbicide tolerant traits, optionally, transgenic traits.

### Example 12: Rearranging Pericentromeric Chromosomal Regions Associated with Yield or Other Agronomic Traits in Plant Cells

In this example, chromosomal region or segments, including QTLs associated with one or more yield-related agronomic traits (e.g., reduced root lodging, reduced brittle snap, drought tolerance) in crop plants such as corn, soybean, cotton, canola, wheat, rice, sorghum, or sunflower are rearranged (e.g., inversion, translocation) such that those chromosomal regions are in a preferred chromosomal configuration that enables faster trait introgression, reduced linkage drag, optimal linkage disequilibrium compared to control and other breeding enhancements. In an embodiment, such chromosomal segments are translocated to a preexisting transgenic locus containing one or more disease, insect and/or herbicide tolerant traits, optionally, transgenic traits.

Alternatively, large chromosomal inversions can be used to close recombination in a chromosomal fragment comprising several genes/QTLs of interest or even the entire chromosome.

### Example 13: Targeted Interspecies and Intergenomic Centromeric Chromosomal Exchange in Plant Cells

In an aspect, targeted CRISPR-Cas mediated DNA breaks promote wide hybridization (hybridization between cultivated species and their wild relatives). For example, a disease resistant QTL from Sorghum can be targeted for interspecific (i.e., interspecies) chromosomal translocation to a related or distant crop species. Increasing the availability of gene pool of a crop is useful to enhance tolerance of major biotic and abiotic stresses and improve the quality characteristics of the plant through plant breeding. For example, targeted genetic segments can be translocated among related brassica species such as B. carinata, B. juncea, B. oleracea and B. campestris.

In another aspect, one of the hurdles in breeding improvement of a crop is the lack of variability at the genetic/phenotypic level within a given species. Therefore, wide hybridization is a tool to create additional variability for broadening the germplasm diversity. The methods disclosed herein provide approaches to target specific chromosomal translocations, rearrangements or other chromosomal segment (large DNA fragment) transfer or integration into a host chromosome to create genetic diversity from a wide or distant donor chromosome. In an aspect, the donor chromosome is an alien chromosome and in an aspect is not transmitted to one or more subsequent generations.

In another aspect, targeted chromosomal exchange that are intergenera can be accomplished through CRISPR-Cas mediated DNA breaks. For example, desirable chromosomal regions can be translocated, exchanged or otherwise transferred between the donor and recipient genera-wheat and rye genomes. In an aspect, difficulties encountered with zygote formation, zygote development and F1 seedling development in wide cross hybrids can be minimized by targeted CRISPR-Cas approaches. For example, by increasing the recombination frequencies of such distant chromosomes, the efficiency of zygote formation and seedling development can be improved.

### Example 14: Duplication of a Subtelomeric and/or Pericentromeric Chromosomal Region in Plant Cells to Improve Trait Integration in Hybrid Crops

Genes, alleles or QTLs can be recessive or semi-dominant and require two or more copies of the gene or QTL to obtain the desired trait. In hybrid crops this requires that the gene or QTL is introgressed into both the male and female parents. This introgressed region can bring additional genomic regions that results in linkage drag. If the causal gene is known, then a plasmid vector carrying the gene necessary for the desired trait can be used as a template to add an additional copy to a parent using CRISPR or traditional transgenic approaches. However, if two or more copies of a QTL are needed, the QTL region can be duplicated using DSB technology. To the extent a QTL for the desired trait is located on a homologous chromosome, e.g., CRISPR-Cas (or any double strand break inducing agent) can be used to make a unique single double stranded break in both chromosomes, one distal to the QTL region on one chromosome and one proximal to the QTL on the other chromosome, and optionally the duplicated segment can be in the pericentromeric region to have the beneficial QTL fixed for breeding purposes. By having the QTL region duplicated on one parent, breeders need not have to select for this trait in one parent allowing for increased genetic gain being obtained in a crop, including for example hybrid or varietal crops.

As described in Example 11, a QTL region can be relocated from one chromosome to another non-homologous chromosome through one or two consecutive translocations or more specifically centromeric inversions. This approach can also be used to create duplicated regions at a desired location. In certain embodiments, the first step of relocating a QTL region to the desired location requires using CRISPR-Cas to induce a double-stranded break in each of the non-homologous chromosomes. To create the first variant CRISPR-Cas induces a double stranded break proximal to the location of where the second copy of the QTL will be positioned in the new desired location. Then the second variant is created by first executing step 1 as previously described for a translocation between non-homologous chromosomes; however, this second CRISPR-Cas induced double-stranded break is distal to the location of where the relocated QTL in the first recovered translocation (first variant) would be located and a different CRISPR-Cas induced double-stranded break proximal of the QTL in a non-homologous chromosome. If needed for certain instances, after a translocation is obtained, a second translocation may be needed between homologous chromosomes to restore the structure of the distal end of chromosome. These separate recovered translocations from variant 1 and variant 2 can then be combined using standard breeding approaches to the extent that the two locations of the translocations are sufficiently distanced apart.

In certain embodiments, for chromosomal segment duplication to occur at a high-enough frequency, the target sites would have to be different in the homologous chromosomes. Thus, SNPs in target sites would be needed if such SNPs are not already present in the target sites for cleaving. These unique target sites can be created by introduction of NHEJ-mediated mutations (small deletions or insertions) into target sites, followed by crossing the edited plants with WT plants and then select progeny plants with both WT and edited chromosomes with those mutations. This will allow using different gRNAs to cut only one of the homologous chromosomes. This example further demonstrates that target site uniqueness can be engineered into a QTL or a chromosomal segment to increase frequency of duplication such that both target sites do not get cut at the same time, which may result in reduced recovery of duplicated chromosomal segments.

QTL duplication (or any duplication of a gene or an allele or a SNP or any genetic element where multiple copies are desired) can be accomplished by the methods described herein. Polyploid crops such as wheat, canola and others benefit from chromosomal segment duplication because tandemly duplicated or co-location of alleles that are needed in homozygous state or in multiple copies, simplifies breeding. In addition, crops where transgenic traits are prevalent (e.g., corn, cotton and soybeans), breeding of QTLs and introgressing transgenic traits are complex. Engineering targeted duplication of favorable alleles or QTLs creates more breeding approaches where the desired traits need not be present on both the female and male parents. Instead, breeders can focus on trait introgression of transgenic traits and reduce linkage drag, thereby increasing overall genetic gain.

This example demonstrates QTL duplication using Cas endonuclease. However, QTL duplication can be performed by other techniques based on the successful demonstration that a large chromosomal segment can be inverted in a complex commercial elite crop plant-maize.

### Example 15: Chromosomal Inversions to Improve Breeding Methods and Increase Recombination Frequency in Plants

Double strand breaks (DSBs) in chromosomal DNA are common and happen e.g., due to exposure to exogenous agents (radiation, chemical agents, etc.) or as the result of normal metabolic cell processes, which are not considered targeted or site-specific double strand breaks introduced by human intervention. DSBs are usually repaired restoring the original order of the chromatin using either non-homologous end joining (NHEJ) or homology directed repair (HDR) pathways. As multiple DSBs can occur simultaneously, in some cases their repair may result in large chromosomal aberrations such as deletions, duplications, and inversions (if two breaks occurred in the same chromosome), or in reciprocal or non-reciprocal translocations and chromosomal fusions involving fragments of different chromosomes. Although infrequent, these types of random chromosomal rearrangements (CRs) are known to occur spontaneously and considered important elements of plant adaptation and speciation as part of the evolutionary process.

Development of site-directed nuclease technologies provided new and efficient methods for targeted gene modifications. However, precise manipulation of large chromosomal fragments that can provide new advantages for crop breeding programs have not been fully recognized.

Crossing over (also referred to as crossover) is the natural mechanism allowing for exchange of genetic information between homologous chromosomes (genetic recombination) and is the main source of diversity and genetic gain in plant breeding programs. Genetic recombination allows a breeder to select for genetic gain because it creates new combinations of alleles at genetically linked loci or genetically distant loci. For example, one line has a favorable allele at a first locus, and an unfavorable allele at a second, and the other line has an unfavorable allele at the first locus and a favorable allele at the second, and the alleles are genetically linked on the same chromosome or as part of a linkage group. Then, crossing the two lines allows the breeder to select offspring of the F1 carrying the favorable alleles at both loci as the result of genetic recombination between the chromosomes of the parents. This selection depends on a number of factors and often requires screening of thousands of progenies to select the one with desired combination of alleles. This screening is inefficient and impedes the progress of genetic gain. Further, the availability of recombinogenic loci limits the generation of genetic diversity in a breeding pool or a breeding population.

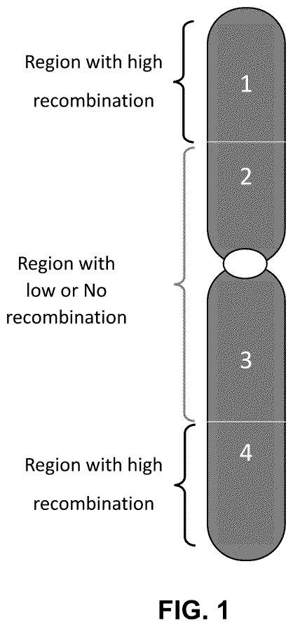

However, a large portion of a genome (chromosome) associated with pre-centromeric regions are known to be either closed for crossovers or demonstrate impractically low frequencies (FIG. 1). As a result, in many crop species, up to 60% of the genomes (chromosomal regions) are excluded from recombination and therefore, are not exploited for breeding experiments. This lack of availability of a substantial portion of the genome significantly limits germplasm diversity and genetic gain potential, as measured by for example, grain yield or other reproductive yield parameters or other agronomic parameters.

Chromosome engineering is based on site-specific DNA break technology and generation of targeted DSBs that can be repaired resulting in various chromosomal rearrangements, such as translocations or inversions. CRISPR-Cas systems provide one such tool to engineer chromosome. These rearrangements change chromosomal architecture and may open up or close large fragments to different chromosomes or chromosomal regions for recombination-based breeding methods.

An experiment demonstrating an approximately 76 megabase long pericentric inversion that naturally occurred in chromosome 2 of one elite genotype of maize (PH1V5T). Two thousand maize immature embryos were transformed using particle bombardment delivery of two in vitro assembled Cas9-gRNA RNP complexes targeting selected sites at the borders of the inversion, and three plasmid vectors carrying a selectable marker gene (NpiII) and two morphogenic transcription factors: maize Baby boom (Bbm) and maize Wuschel2 (Wus2).

The inversion happened in two out of 1500 regenerants analyzed. This result demonstrates feasibility of large chromosomal inversions using CRISPR-Cas DSB technology with practical frequencies. Experiment was then repeated using Agrobacterium-mediated delivery and 18 plants with inversion were identified among 2300 regenerants analyzed.

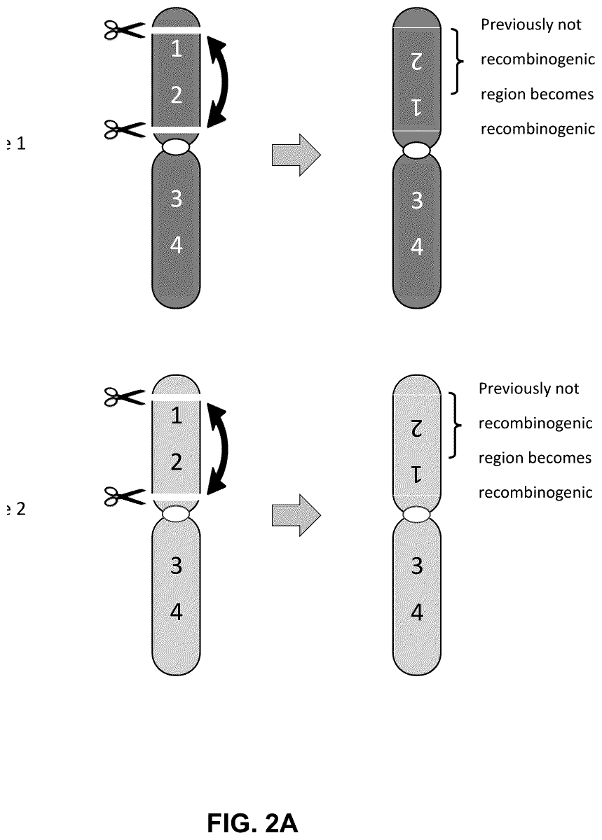

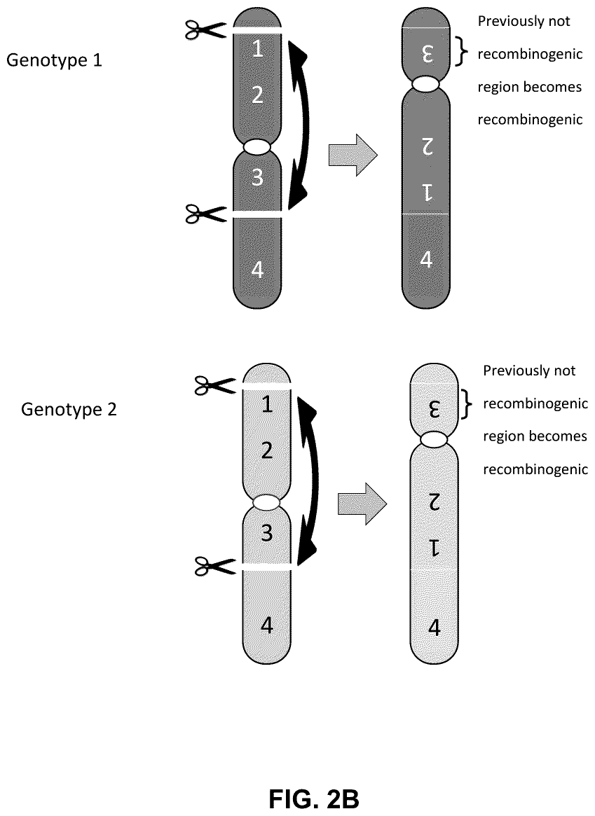

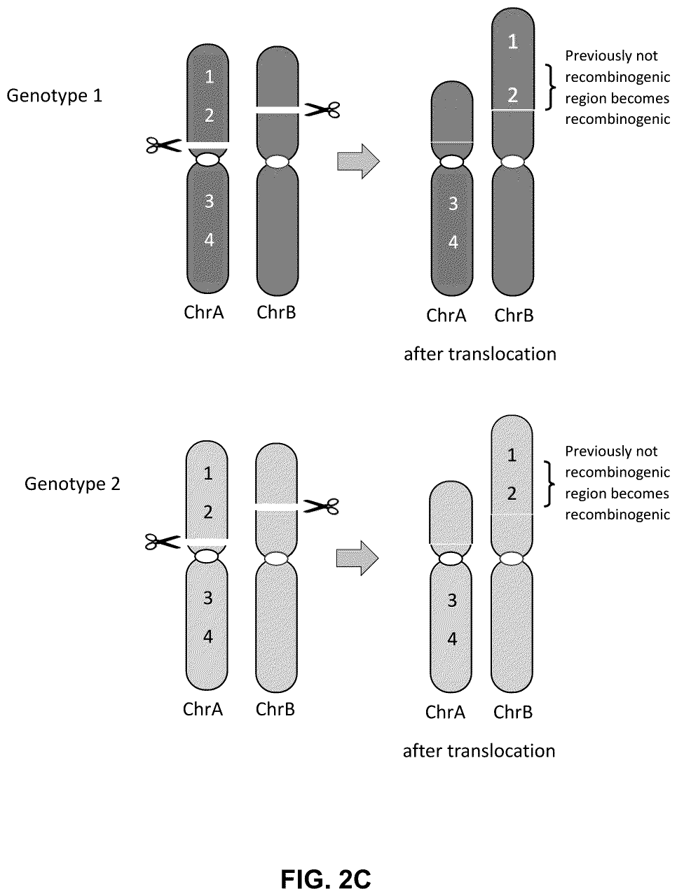

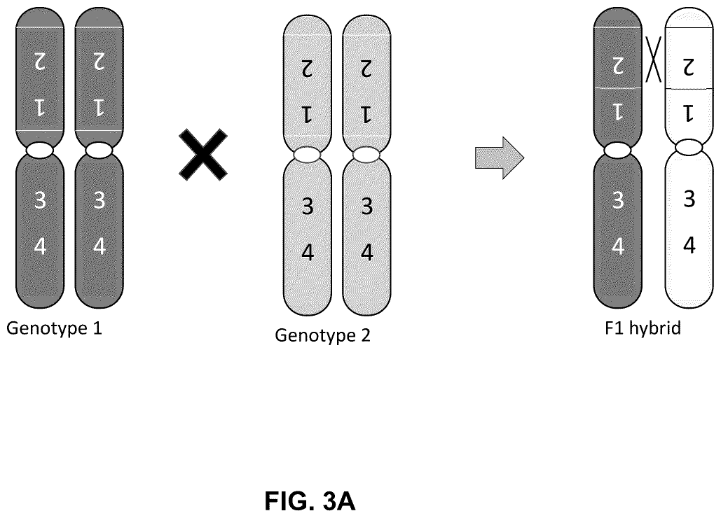

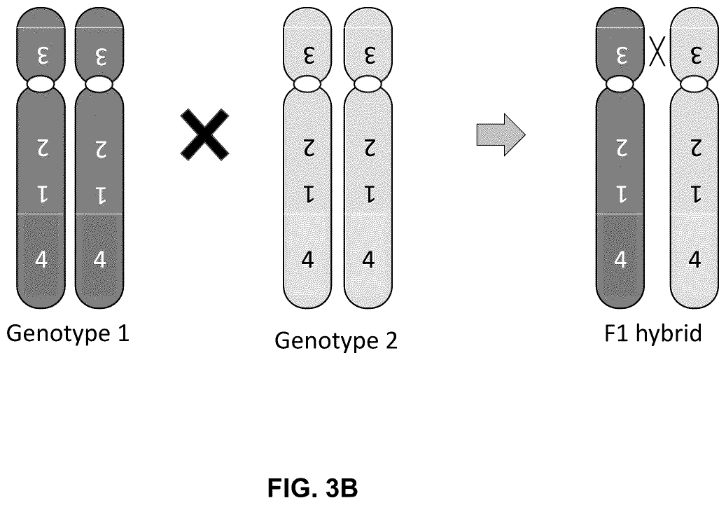

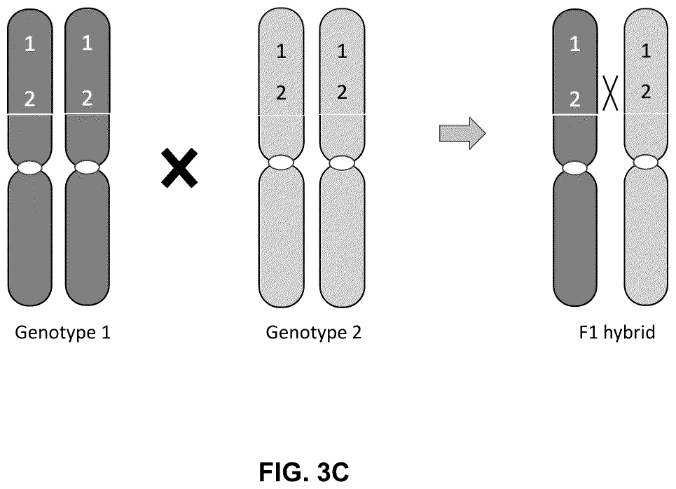

In an illustrated embodiment shown, large para-centric (FIG. 2A) or peri-centric (FIG. 2B) inversions including the large part of chromosomal arm between centromeric and telomeric regions results in re-orientation of the arm and positioning of a previously pre-centromeric region close to telomere. In addition, translocation between two homologous or homeologous chromosomes can lead to changing pre-centromeric region position (FIG. 2C). This positional change of a large pre-centromeric chromosomal region will open this genomic region for recombination allowing generation of new combinations of alleles at genetically linked loci allowing a breeder to select for genetic gain (FIG. 3). In addition, such rearrangements may place previously highly recombinogenic sub-telomeric region close to centromere resulting in closing it for further recombination, thus preserving the existing set and order of allelic combinations, where such allelic combinations are desirable for breeding—e.g., collection of QTLs or transgenic events for forward breeding or trait introgression.

The illustrated embodiments shown in FIGS. 2 and 3 (describing the paracentric and pericentric inversions, and chromosomal translocations) can be performed on one or more of chromosomal arms of any plant species opening the entire genome or a substantial portion of the genome for recombination and thereby provide an increase in diversity for genetic gain. Optionally, once recombination experiments are completed (i.e., after a desirable set of alleles are generated), the modified chromosomal arm can be rearranged to restore its original orientation and position, allowing a breeder to perform trait introgression and selection of the best performing genotypes. Such type of chromosomal architecture restoration after recombination can help with genomic stability during mitosis, meiosis and cell divisions generally to preserve appropriate chromosomal segregation.

### Example 16: Modulating Chromatin Compaction to Improve Breeding Methods and Increase Recombination Frequency in Plants Through Chromosomal Manipulation

Targeted DNA breaks (including nicks and double strand breaks) at regions of low recombinogenic in the chromosome e.g., centromeric and pericentromeric regions to induce inversions and translocations are discussed in one or more Examples herein. To further enhance the recombination frequency of such regions either before or after the inversion/translocation event has occurred, agents or factors to modulate chromatin compaction are described in this Example. In an embodiment, Histone deacetylase (HDAC) inhibitors such as trichostatin A (TSA) is used to modulate chromatin, for example, loosening chromosomal packaging by inhibiting histone deacetylase.

In an embodiment, epigenetic modifications of chromosomal DNA and histones are capable of modulating plant chromatin architecture to enable recombination of inverted or translocated pericentromeric regions. These epigenetic modifications include DNA methylation and histone acetylation. Positive charge of the histones is reduced by the acetylation of lysine residues on the N-terminal tails of histones. This charge alteration impacts the histone-histone and histone-DNA interaction, thereby modulating the accessibility of DNA to the chromatin-binding proteins, including those that are involved in recombination. Generally, an increase in the acetylation of histones promoted more open chromatin state and a decrease in acetylation (hypoacetylation) of histones is indicative of a more silenced chromosomal state.

In this Example, modulation of chromatin compaction in both somatic and germ cells is contemplated. In an embodiment, upon a successful targeted pericentromeric inversion or translocation of a low recombinogenic region to a high recombinogenic chromosomal location, recombination frequency is enhanced by modulating chromatin compaction. During recombination, ‘crossover events’ (COs) that generate a chromosome containing long DNA sequences from each of the contributing chromosomes are enhanced compared to the ‘non-crossover events’ (NCOs) or ‘gene conversions’, where only a small section of the chromosomal DNA is exchanged between the homologous chromosomes.

Other methods to enhance centromeric inversions include for example, increased temperature during targeted DNA breaks and methods to reduce NHEJ and promote strand invasion for targeted crossover events. Cell cycle modulating agents are also suitable to increase targeted, stable and inheritable centromeric inversions described herein. In certain embodiments, the centromeric chromosomal rearrangements including inversions and translocations described herein are enhanced by using one or more factors involved in recombination. Examples include Spol1 polynucleotide or polypeptide (exogenous or endogenous), a Rec8 (aka Afd1 in maize) (exogenous or endogenous) polynucleotide or polypeptide, an OSD1-1A (exogenous or endogenous) polynucleotide or polypeptide, and an OSD1-3A (exogenous or endogenous) polynucleotide or polypeptide, and RecQ helicases.

1. A method for producing an increased recombinogenic parental line of a crop plant, the method comprising: identifying and targeting a low-recombinogenic pericentromeric chromosomal segment in a chromosomal arm of a crop plant cell to rearrange to a higher recombinogenic state in the chromosomal arm of the plant cell by introducing one or more site-specific DNA breaks at one or more target sites in the chromosomal segment and obtaining the modified chromosomal arm comprising the rearranged chromosomal segment that is rearranged such that the rearranged chromosomal segment is capable of increased recombination compared to a control plant cell not comprising the rearranged chromosomal segment, wherein the recombinogenic parental line is stable and the rearranged chromosomal segment is heritable in one or more progeny population.

2. The method of claim 1, wherein the pericentromeric chromosomal segment is rearranged such that a portion of the chromosomal segment that was positioned closer to a centromeric region in an unmodified state is closer to a telomere of the chromosomal arm in the rearranged or modified state.

3. The method of claim 1, further comprising generating the stable, modified chromosomal arms of more than one chromosome in two or more parental lines of a breeding population for a crop breeding program, such that the modified chromosomes are recombination compatible in a breeding cross.

4. The method of claim 1, wherein the DNA breaks are targeted double strand breaks introduced by a site-specific nuclease selected from the group consisting of CRISPR-Cas nucleases, TALENs, meganucleases and zinc finger nucleases.

5. The method of claim 1, wherein the chromosomal rearrangement is an inversion or a translocation.

6. The method of claim 1, wherein the chromosomal rearrangement is within the same arm of the chromosome.

7. The method of claim 1, wherein the rearranged or translocated chromosomal segment comprises at least about 50 kb, 75 kb, 100 kb, or 500 kb of contiguous bases.

8. The method of claim 1, wherein the rearranged or translocated chromosomal segment is larger than about 1 Mb, 5 Mb, 10 Mb, 15 Mb, 20 Mb, 25 Mb, 50 Mb or 100 Mb.

9. The method of claim 1, wherein the rearranged or translocated chromosomal segment comprises one or more QTLs or one or more favorable alleles associated with an agronomic trait that were previously not associated with such traits due to low observable recombination frequency.

10. The method of claim 1, wherein the modified chromosome or sister chromatids comprising the modified chromosomes are capable of segregating during cell division.

11. The method of claim 1, wherein the plant cell is a somatic cell.

12. The method of claim 1, wherein the recombination is increased during meiotic or mitotic cell division.

13. The method of claim 1, wherein the crop plant is selected from the group consisting of corn, soybean, cotton, canola, sorghum, wheat, rice, sunflower, and alfalfa.

14. A method of increasing recombinogenic potential of a crop breeding population, the method comprising:
a) inverting a first pericentromeric chromosomal segment of at least one chromosomal arm of a first breeding line such that the inverted chromosomal segment is positioned closer to the telomeric side of the chromosomal arm;
b) inverting a second pericentromeric chromosomal segment of at least one chromosomal arm of a second breeding line such that the inverted chromosomal segment is positioned closer to the telomeric side of the chromosomal arm;
c) obtaining a breeding pair comprising the first breeding line having the first pericentromeric chromosomal segment inverted and the second breeding line having the second pericentromeric chromosomal segment inverted;
d) crossing the first breeding line and the second breeding line with inverted at least two inverted chromosomal segments; and
e) obtaining progeny as a result of a cross between the first breeding line and the second breeding line with inverted para-centromeric chromosomal segments, wherein the progeny is genetically unique compared to a progeny obtained from crossing breeding lines not comprising the inverted chromosomal segments.

15. The method of claim 14, wherein the crop is maize.

16. The method of claim 14, wherein the chromosomal segment is at least 50 kb.

17. The method of claim 14, wherein the chromosomal segment is larger than 1 Mb.

18. The method of claim 14, wherein the breeding population comprises about 10% to 100% of the chromosomes that comprise at least one modified chromosomal arm that includes the inverted pericentromeric chromosomal segment.

19. The method of claim 14, wherein the inverted chromosomal segments are accomplished through targeted site-specific double strand breaks with a site-specific nuclease.

20. The method of claim 14, wherein the inverted chromosomal segments are present in homologous chromosomes.

21. The method of claim 1, wherein the rearrangement or translocation results in unequal inversion of the chromosomal segment.

22. A method for relocating a pericentromeric segment of a chromosome in a crop plant, the method comprising:
a) introducing a first targeted site-specific break at a chromosomal arm of the chromosome, wherein the first break is present at a pericentromeric segment of the chromosomal arm and introducing a second targeted site-specific break at a chromosomal arm of the chromosome, wherein the second break is closer to a telomeric region of the chromosomal arm;
b) obtaining a plant with a desired translocation, wherein the pericentromeric segment is rearranged towards the telomeric region of the chromosomal arm;
c) generating progeny plants with the desired rearrangement; and
d) utilizing the progeny plants in a crop breeding program for increasing genetic gain.

23. A method for trait introgression from a wild race to a cultivated genotype or from a genotype with the trait of interest to an elite genotype not carrying the trait, through a paracentric chromosomal inversion breeding approach, the method comprising
i) generating a recipient plant comprising a modified genotype, wherein the recipient plant comprises at least one chromosome wherein a paracentric inversion of one of the chromosomal arms is introduced by a human induced site-specific nuclease activity;
ii) obtaining a wild relative donor plant, wherein the wild relative donor plant comprises a trait of interest and is capable of being crossed with the recipient plant;
iii) performing a cross between the modified recipient plant and the donor plant to produce one or more progeny plants; and
iv) allowing chromosomal DNA transfer to occur between the donor and the recipient plant species, thereby engineering inter-chromosomal translocation and/or QTL containing DNA transfer between the donor and the recipient plants; and
v) selecting the progeny plant comprising the trait of interest obtained from the donor plant.

24. The method of claim 1, wherein the crop plant that comprises the paracentric or paracentromeric inversion or translocation is fertile and is capable of setting seeds.

25. A method for paracentric inversion of a chromosomal segment of more than one chromosome in a crop plant cell to enhance genetic recombination between homologous chromosomes during meiosis and increase genetic diversity wherein the chromosome comprises a first and second target site in the paracentromeric region, the method comprising:
a) introducing to the chromosome a Cas endonuclease and a first and second guide RNA, wherein the Cas endonuclease and the first and second guide RNA form a first and second complex, respectively and providing one or more oligonucleotides with substantial sequence similarity to one or more chromosomal ends at two different or distinct double strand break regions; wherein each of the first and second complexes recognizes, binds to, and cleaves the first and second target sites, respectively;
b) incubating the cell under conditions that allow for repair of the two cleavages at the two target sites, wherein the repair results in the inversion of the segment; and
c) validating the inversion by genotype or phenotype of the cell, or an organism that comprises the cell;
wherein the segment comprises at least one million contiguous bases, wherein the first and second target sites flank the segment.

26. A method of increasing the efficiency of interspecific and/or intergeneric chromosomal DNA transfer, the method comprising (i) providing a donor plant species and a recipient plant species, wherein at least the recipient or the donor plant species comprise a paracentric or a pericentric inversion of at least one of the chromosomes; (ii) optionally introducing targeted double strand or single strand DNA breaks in the chromosomal regions of the donor and/or the recipient plant species such that large chromosomal fragments are targeted and providing one or more oligonucleotides with substantial sequence similarity to one or more chromosomal ends at two different or distinct double strand break regions; and (iii) allowing chromosomal DNA transfer to occur between the donor and the recipient plant species, thereby engineering interspecific and/or intergeneric chromosomal DNA transfer between the donor and the recipient plant species.

27. A method of reducing recombination frequency within a chromosomal segment to preserve linkage disequilibrium of one or more favorable alleles or SNPs or traits of interest in a chromosome of a crop plant, the method comprising introducing site-specific double strand breaks in at least two distant target sites of a chromosomal region, wherein the chromosomal region comprises a human introduced paracentric inversion and providing one or more oligonucleotides with substantial sequence similarity to one or more chromosomal ends at two different or distinct double strand break regions, where in the targeted double strand breaks result in an reinversion or a rearrangement of the previously translocated chromosomal segment to its original orientation such that the reinverted or rearranged chromosomal segment does not recombine or recombines at a lower frequency compared to a control plant during meiosis, wherein the reinverted or rearranged chromosomal segment comprises newly acquired or created alleles or desirable haplotypes due to increased recombination from the previous generation.

28. The method of claim 27, wherein the inverted or rearranged chromosomal segment is in the same chromosome as the original pre-inversion/rearrangement chromosome.

29. The method of claim 27, wherein the inverted or rearranged chromosome is in a heterologous or non-homologous chromosome.

30. The method of claim 1, further comprising providing to the crop plant cell at least one morphogenic factor.

31. The method of claim 1, wherein at least one morphogenic factor is BBM or WUS.

32. The method of claim 1, wherein the DNA break inducing agent is CRISPR-Cas and is provided directly to the cell as a protein.

33. The method of claim 1, wherein the DNA break inducing agent is CRISPR-Cas and is provided directly to the cell as an RNA molecule.

34. The method of claim 1, wherein the DNA break inducing agent is CRISPR-Cas and is transfected/electroporated to plant protoplasts or delivered to intact plant cells with cell walls using particle bombardment.

35. The method of claim 1, wherein the DNA break inducing agent is CRISPR-Cas and is provided directly to the crop plant cell by Agrobacterium-mediated delivery.

36. An engineered crop plant cell comprising a human introduced, targeted modified chromosomal arm of a crop plant chromosome, wherein a low recombinogenic para-centromeric chromosomal segment is present in a heterologous location of a higher recombinogenic state in the chromosomal arm of the plant cell and exhibits increased recombination compared to a control plant cell not comprising the rearranged chromosomal segment, during meiosis or mitosis.

37. The crop plant cell of claim 36 is maize.

38. The crop plant cell of claim 36 is selected from the group consisting of wheat, rice, canola, sunflower, cotton and sorghum.

39. A crop plant seed comprising the cell of claim 36.

40. The engineered crop plant cell of claim 36 further comprising a reinverted paracentric chromosomal segment.

41. The engineered crop plant cell of claim 36, wherein the paracentric chromosomal segment is inverted, thereby translocating to the heterologous location.

42. A crop plant comprising the cell of claim 36.

43. A maize plant seed comprising the cell of claim 36.

44. The maize plant seed of claim 43 is an inbred.

45. The maize plant seed of claim 43 is an F1 progeny.

46. The method of claim 1, wherein the chromosomal rearrangement is not a Robertsonian translocation.

47. The method of claim 1, wherein the high recombinogenic state is sub-telomeric.

48. The method of claim 1, wherein the recombinogenic potential of crop plant is further enhanced by modulating chromatic compaction of the modified parental line.

49. The method of claim 1, wherein the chromosomal rearrangement in increased by temperature modulation.

50. The method of claim 1, wherein the chromosomal rearrangement in increased by using a chromosome structure modulation agent.
